(19) United States
(12) Patent Application Publication
(10) Pub. No.: US 2010/0022596 A1
(43) Pub. Date: Jan. 28, 2010

(54) EPOTHILONE ANALOGUES

(76) Inventor: Karl-Heinz Altmann, Reinach (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/518,159

(22) PCT Filed: Dec. 12, 2007
(86) PCT No.: PCT/EP2007/010846

§ 371 Date: Jun. 8, 2009

(30) Foreign Application Priority Data
Dec. 14, 2006 (EP) 06126141.8

(51) Int. Cl. A61K 31/4439; C07D 277/30; C07D 417/14; A61K 31/427; A61P 35/00

(52) U.S. Cl. 514/338; 548/204; 546/269.7; 514/365

(57) ABSTRACT

The invention relates to analogues of epothilones, uses and methods of making the same.

The invention is related to analogues of epothilone A.

More particularly, the invention is directed to analogs where the epoxide ring at C13/C14 of epothilone is replaced by one of a 2-substituted-2,5-dihydro-oxazole or a 2-substituted thiazolidine.

### BACKGROUND TO THE INVENTION

Epothilones are macrolide compounds which find utility in the pharmaceutical field. For example, Epothilones A and B having the structures below have been found to exert microtubule-stabilizing effects and hence cytotoxic activity against rapidly proliferating cells, such as, tumor cells or other hyperproliferative cellular disease.

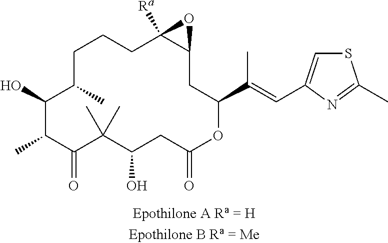

### DETAILED DESCRIPTION OF THE INVENTION

One aspect of the invention is directed to compounds represented by one of the following formulae:

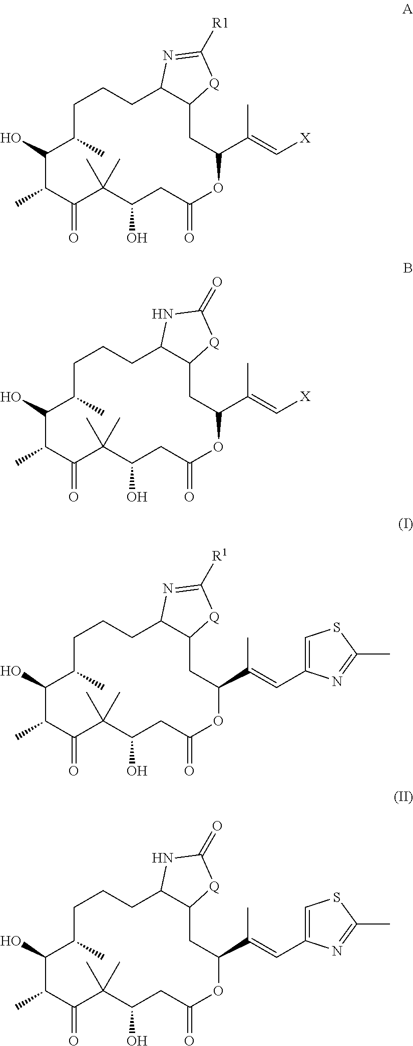

where Q is a radical selected from O or S; and
R¹ may be selected from hydrogen, halogen, hydroxy, hydrocarbyl, trifluoromethyl, cyano, nitro, amidino, —B(OH)₂, ═NR², —OR², —SR², —C(O)R², —C(O)OR², —OC(O)R², —N(R²)R³, —C(O)_{K}N(R²)R³, (CR⁵R⁶)ⱼ—S(O)ₗR², —C(R²)₃ and R⁴;
R² and R³ are each independently hydrogen or are selected from C₁₋₆ alkyl, —(CR⁵R⁶)ⱼ-carbocyclyl and —(CR⁵R⁶)ⱼ-heterocyclyl, any of which is optionally substituted with 1, 2, 3, 4 or 5 substituents independently selected from halogen, hydroxy, C₁₋₆ alkyl, trifluoromethyl, cyano, nitro, amino and amidino;
Each R⁴ and X may be the same or different and are both independently selected from C₁₋₆ alkyl, C₂₋₆ alkenyl, C₂₋₆ alkynyl, C₁₋₆ alkoxy, —(CR⁵R⁶)ⱼ-carbocyclyl and —(CR⁵R⁶)ⱼ-heterocyclyl, aryl, heteroaryl, amino any of which is optionally substituted with 1, 2, 3, 4 or 5 substituents independently selected from halogen, hydroxy, C₁₋₆ alkyl and C₁₋₆ alkoxy;
each R⁵ and R⁶ may be the same or different and are both independently selected from a bond, hydrogen, halogen, hydroxy and amino;
j is 0, 1, 2, 3, 4, 5, 6 or 7;
k is 0 or 1;
l is 0, 1 or 2
or pharmaceutically acceptable salts, esters, solvates, N-oxides or prodrugs thereof.

### DEFINITIONS

### Hydrocarbyl

The term “hydrocarbyl” as used herein includes reference to a moiety consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19 or 20 carbon atoms. Examples of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₂₋₆ alkenyl; C₂₋₆ alkynyl; C₁₋₆ alkoxy; each of which may be substituted by aryl (e.g. benzyl) or by cycloalkyl (e.g cyclopropylmethyl); cycloalkyl (e.g. cyclopropyl, cyclobutyl, cyclopentyl or cyclohexyl); aryl (e.g. phenyl, naphthyl or fluorenyl) and the like.

### Alkyl

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, alkyl may have 1, 2, 3 or 4 carbon atoms.

### Alkenyl

The terms “alkenyl” and “C₂₋₆ alkenyl” as used herein include reference to a straight or branched chain alkyl moiety having 2, 3, 4, 5 or 6 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

### Alkynyl

The terms “alkynyl” and “C₂₋₆ alkynyl” as used herein include reference to a straight or branched chain alkyl moiety having 2, 3, 4, 5 or 6 carbon atoms and having, in addition, at least one triple bond. This term includes reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered non-aromatic ring or ring system and, in particular, a 5- or 6-membered non-aromatic ring, which may be fully or partially saturated. A carbocyclic moiety is, for example, selected from cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like.

### Cycloalkyl

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

### Heterocyclyl

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, heterocyclyl includes a 3- to 10-membered non-aromatic ring or ring system and more particularly a 5- or 6-membered ring, which may be fully or partially saturated.

A heterocyclic moiety is, for example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

### Heteroaryl

The term “heteroaryl” as used herein includes reference to an aromatic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein includes reference to F, Cl, Br or I. In a particular class of embodiments, halogen is F or Cl, of which F is more common.

### Amino

The term “amino” as used herein includes reference to moieties of the general structure —N(R²)R³ and particularly includes —NH₂ and —NHR², where R² and R³ are as hereinbefore defined.

### Amidino

The term “amidino” as used herein includes reference to moieties of the general structure —C(NH)NH₂ and derivatives thereof, in particular, those in which a hydrogen is replaced by alkyl, (e.g. methyl or ethyl) or hydroxy.

### Halogen

The term halogen as used herein includes fluoro, chloro bromo and iodo. Fluoro may be mentioned in particular.

### Substituted

The term “substituted” as used herein in reference to a moiety means that one or more, especially up to 5, more especially 1, 2 or 3, of the hydrogen atoms in said moiety are replaced independently of each other by the corresponding number of the described substituents.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled man.

Where steric issues determine placement of substituents on a group, the isomer having the lowest conformational energy may be preferred.

### Independently

Where two or more moieties are described as being “each independently” selected from a list of atoms or groups, this means that the moieties may be the same or different. The identity of each moiety is therefore independent of the identities of the one or more other moieties.

### Pharmaceutically Acceptable

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Salts are especially the pharmaceutically acceptable salts of compounds of Formula (I) (or exemplary formula thereof), especially if they are forming salt-forming groups.

Salt-forming groups are groups or radicals having basic or acidic properties. Compounds having at least one basic group or at least one basic radical, for example amino, a secondary amino group not forming a peptide bond or a pyridyl radical, may form acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethanesulfonic acid, or aromatic sulfonic acids, for example benzene-, p-toluene- or naphthalene-2-sulfonic acid. When several basic groups are present mono- or poly-acid addition salts may be formed.

Compounds having acidic groups, a carboxy group or a phenolic hydroxy group, may form metal or ammonium salts, such as alkali metal or alkaline earth metal salts, for example sodium, potassium, magnesium or calcium salts, or ammonium salts with ammonia or suitable organic amines, such as tertiary monoamines, for example triethyl-amine or tri-(2-hydroxyethyl)-amine, or heterocyclic bases, for example N-ethyl-piperidine or N,N′-dimethylpiperazine. Mixtures of salts are possible.

Compounds having both acidic and basic groups can form internal salts.

For the purposes of isolation or purification, as well as in the case of compounds that are used further as intermediates, it is also possible to use pharmaceutically unacceptable salts, e.g. the picrates. Only pharmaceutically acceptable, non-toxic salts may be used for therapeutic purposes, however, and those salts are therefore preferred.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the dose relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

In preferred embodiments of the present invention, R¹ has the formula:

—(CR⁵R⁶)ⱼ—Ar (III)

where each R⁵ and R⁶ may be the same or different and are both independently selected from hydrogen, halogen, hydroxy;
j is 0, 1, 2, 3, 4, 5, 6 or 7; and
Ar is an aromatic moiety, which may be optionally substituted with substituents independently selected from halogen, hydroxy, C₁₋₆ alkyl, C₁₋₆ alkenyl, C₁₋₆ alkynyl, C₁₋₆ alkoxy, trifluoromethyl, cyano, nitro amino and amidino.

The moiety CR⁵R⁶ may be saturated or unsaturated. In the case where the moiety is unsaturated, one of the moieties R⁵ or R⁶ is a bond.

In one class of compounds, Ar is an optionally substituted aromatic or heteroaromatic 5- or 6-membered ring.

Ar may be, for example, phenyl, pyridyl and thiophenyl.

In a further class of compounds, R¹ has the formulae:

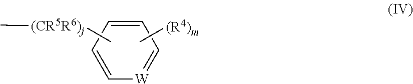

where R⁵, R⁶, j and R⁴ are as hereinbefore described; and

### W is N or C; and

m is 0, 1, 2, 3 or 4.

In a preferred class of compounds of formula IV, the substituents are para to one another, as provided for in the formula below:

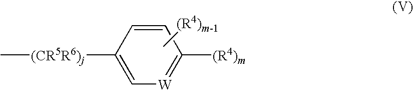

where R⁵, R⁶, j and R⁴ are as hereinbefore described, and

### W is N or C; and

m is 0, 1, 2, 3 or 4.

In a further class of compounds, R¹ has the formulae:

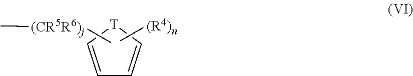

where n is 0, 1, 2 or 3 and T is NH, O or S.

In one particular class of compounds, R⁵ and R⁶ are both hydrogen.

In another class of compounds, j is preferably 0, 1 or 2.

In a further sub-class of compounds, j is 0.

R⁴ is preferably selected from halogen and C₁₋₆ alkyl, of which methyl, ethyl, isopropyl and tert. butyl may be mentioned in particular.

Compounds of the present invention are preferably of trans configuration at the C12/C13 position and have one of the following formulae:

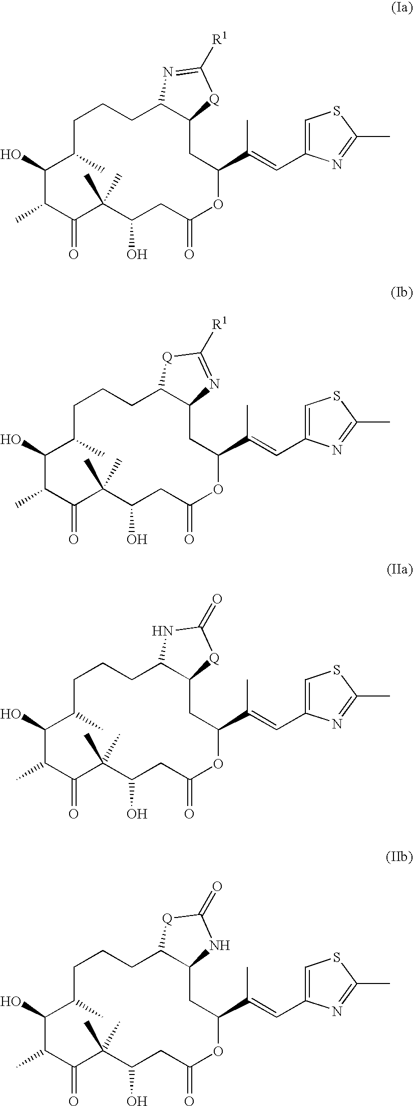

where R¹ and Q are as hereinbefore described.

In one class of compounds, Q is O, providing compounds of formulae:

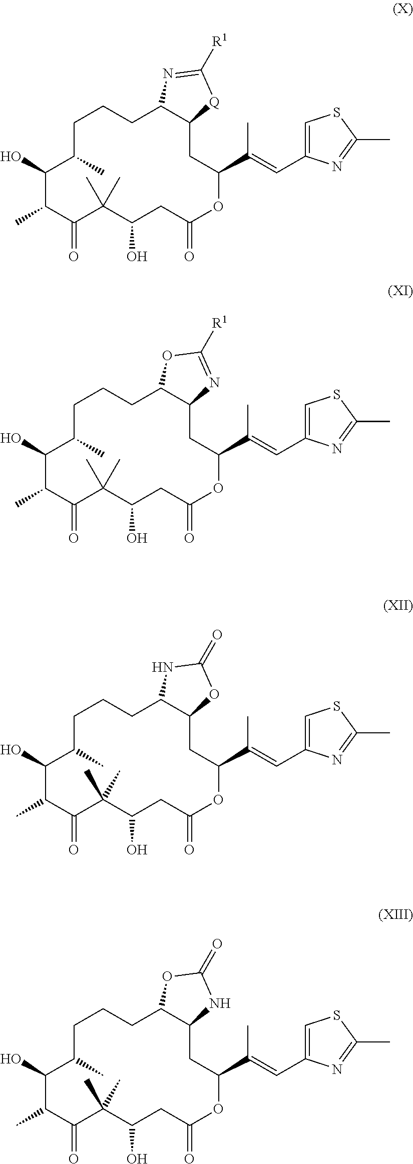

where R¹ is as hereinbefore described.

In one aspect of the present invention, the compounds are metabolically more stable than Epothilone A. In a further aspect, the compounds of the present invention show anti-proliferative activity comparable to that of their natural congener.

Due to the epoxide function at C12/C13, epothilones undergo rearrangement reactions under acidic conditions leading to biologically inactive compounds. As such, the epoxide moiety may be considered to represent a metabolic weak point, which can be hydrolyzed in vivo to produce a biologically inactive diol.

Therefore, the present invention seeks to overcome this problem by replacing the epoxide function with a 5-membered oxazole ring.

The compounds of the present invention, no longer having the expoxide unction, provide a source of epothilone derivatives which are metabolically more stable.

The compounds of the present invention may therefore be more suitable for the development of orally bioavailable anti-cancer drugs.

Compounds of the present invention preferably have biological activity comparable with that of Epothilone A.

In one preferred class of compounds, the epoxide moiety of in Epothilone A is replaced with a 2-substituted 2,5-dihydro-oxazole rings, such as the example shown below:

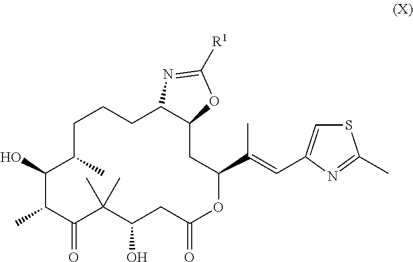

A particular example of this class of compounds, is where the 2,5-dihydro-oxazole ring is substituted by 5 or 6 membered aromatic or heteroaromatic moieties at C2.

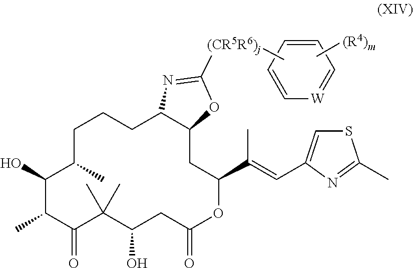

where R⁵, R⁶, j, R⁴, W and m are as hereinbefore described.

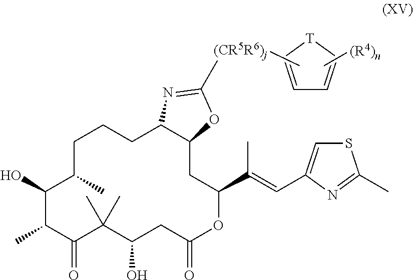

where R⁵, R⁶, j, R⁴, T and n are as hereinbefore described.

These compounds of the present invention are not only more stable towards acidic conditions than their natural congener; moreover, by varying the substituents at C2 of a 2,5-dihydro-oxazole ring, for example, the physicochemical and pharmacological properties of these types of compounds can be modulated.

The hydroxy groups of the compounds described herein may be protected by protecting groups for a hydroxy group.

The term “protecting groups for a hydroxy group” as used herein refers to acid labile protecting groups for a hydroxy group, which groups are known as such. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of hydroxy groups by protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), HoubenWeyl, 4th edition, Volume15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates monosaccharides and derivatives), Georg Thieme Verlag, Stuttgart 1974.

Preferred protecting groups are silyl ethers which are acid labile like tert-butyl-dimethyl-silyl (TBS) ether, triethylsilyl (TES) ether, triisopropylsilyl (TIPS) ether, diethylisopropylsilyl (DEIPS) ether, isopropyldimethylsilyl (IPDMS) ether or thexyldimethylsilyl (TDS) ether.

Another aspect of the invention is directed to an anticancer reagent comprising any of the compounds described above dissolved or suspended in a physiological solvent suitable for administration to a patient. The compound has a concentration within the physiological solvent sufficient to be cytotoxic to a cancer cell.

Another aspect of the invention is directed to a process for killing a cancer cell comprising the step of contacting the cancer cell with a solution containing a cytotoxic concentration of any compound described above.

Furthermore, the present invention pertains to the use of a compound of the present disclosure or a pharmaceutically acceptable salt or a solvate or a hydrate of such a compound, in a method for the treatment of the human or animal body.

Furthermore, the present invention pertains to the use of a compound of the present disclosure, or a pharmaceutically acceptable salt or a solvate or a hydrate of such a compound, for the preparation of a pharmaceutical product for the treatment of a neoplastic disease.

The term “neoplastic disease” relates in particular to liquid tumor diseases, like leukemia, and solid tumor diseases.

The term “solid tumor disease” especially means breast cancer, ovarian cancer, cancer of the colon and generally the Gi tract including gastric cancer, cervix cancer, lung cancer, e.g. small-cell lung cancer and non-small-cell lung cancer, pancreas cancer, renal cancer, glioma, melanoma, head and neck cancer, bladder cancer, thyroid cancer, hepatocellular cancer, prostate cancer and Kaposi's sarcoma.

Moreover, the present invention provides a method for the treatment of a neoplastic disease, which comprises administering a compound of the present disclosure or a pharmaceutical acceptable salt or a solvate or a hydrate of such a compound, in a quantity effective against said disease, to a warm-blooded animal requiring such treatment.

Furthermore, the present invention relates to a pharmaceutical preparation, comprising a compound of the present disclosure, or a pharmaceutical acceptable salt or a solvate or a hydrate of such a compound, and at least one pharmaceutical acceptable carrier that are suitable for topical, enteral, for example oral or rectal, or parenteral administration and that may be inorganic or organic, solid or liquid. There are used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting agents and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving orlyophilising processes, and comprise approximately from 1% to 95%, especially from approximately 1% to approximately 20%, active ingredient(s).

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

Compounds of the present invention are preferably microtubule-stabilizing agents.

They are thus useful in the treatment of a variety of cancers, including (but not limited to) the following; carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Hodgkins lymphoma, non-Hodgkins lymphoma, hairy cell lymphoma and Burketts lymphbma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroidfollicular cancer and teratocarcinoma.

Compounds of the present invention may also inhibit tumor angiogenesis, thereby affecting the growth of tumors. Such anti-angiogenesis properties may also be useful in the treatment of certain forms of blindness related to retinal vascularization, arthritis, especially inflammatory arthritis, multiple sclerosis, restinosis and psoriasis.

Compounds of the present invention may induce or inhibit apoptosis, a physiological cell death process critical for normal development and homeostasis.

Alterations of apoptotic pathways contribute to the pathogenesis of a variety of human diseases. Compounds of the present invention as modulators of apoptosis, will be useful in the treatment of a variety of human diseases with aberrations in apoptosis including cancer (particularly, but not limited to follicular lymphomas, carcinomas with p53 mutations, hormone dependent tumors of the breast, prostrate and ovary, and precancerous lesions such as familial adenomatous polyposis), viral infections (including but not limited to herpesvirus, poxvirus, Epstein-Barr virus, Sindbis virus and adenovirus), autoimmune diseases (including but not limited to systemic lupus erythematosus, immune mediated glomerulonephritis, rheumatoid arthritis, psoriasis, inflammatory bowel diseases and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), AIDS, myelodysplastic syndromes, aplastic anemia, ischemic injury associated myocardial infarctions, stroke and reperfusion injury, arrhythmia, atherosclerosis, toxin-induced or alcohol induced liver diseases, hematological diseases (including but not limited to chronic anemia and aplastic anemia), degenerative diseases of the musculoskeletal system (including but not limited to osteoporosis and arthritis), aspirin-sensitive rhinosinusitis, cystic fibrosis, multiple sclerosis, kidney diseases, and cancer pain.

The compounds of this invention are also useful in combination with known anti-cancer and cytotoxic agents and treatments, including radiation.

The compounds of the present invention can be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents. In particular, compounds of the present invention can be administered for example in the case of tumour therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumour regression, or even chemopreventive therapy, for example in patients at risk.

Therapeutic agents for possible combination are especially one or more antiproliferative, cytostatic or cytotoxic compounds, for example a chemotherapeutic agent or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase, e.g. PKI166, the VEGF receptor tyrosine kinase, e.g. PTK787, or the PDGF receptor tyrosine kinase, e.g. ST1571, a cytokine, a negative growth regulator, such as TGF- or IFN-B, an aromatase inhibitor, e.g. letrozole or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel, discodermolide or an epothilone, alkylating agents, antineoplastic antimetabolites, such as gemcitabine or capecitabine, platin compounds, such as carboplatin or cisplatin, anti-angiogenic compounds, gonadorelin agonists, anti-androgens, bisphosphonates, e.g. AREDIA or ZOMETA, and trastuzumab. The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Compounds of the present invention may also have prodrug forms. Any compound that will be converted in vivo to provide the bioactive agent is a prodrug within the scope and spirit of the invention. For example compounds of the present invention may form a carboxylate ester moiety. The carboxylate esters are conveniently formed by esterifying any of the carboxylic acid functionalities found on the disclosed ring structure (s).

Various forms of prodrugs are well known in the art. For examples of such prodrug derivatives, see: a) Design of Prodrugs, edited by H. Bundgaard, (Elsevier, 1985) and Methods in EnzmmologY, Vol. 42, p. 309-396, edited by K. Widder, et al. (Academic Press, 1985); b) A Textbook of Drug Design and Development, edited by Krosgaard-Larsen and H. Bundgaard, Chapter 5, “Design and Application of Prodrugs,” by H. Bundgaard, p. 113-191 (1991); c) H. Bundgaard, Advanced Drug Delivery Reviews, 8, 1-38 (1992); d) H. Bundgaard, et al., Journal of Pharmaceutical Sciences, 77,285 (1988); and e) N. Kakeya, et al., Chem Phar Bull, 32,692 (1984).

It should further be understood that solvates (e.g., hydrates) of the compounds of the present invention are also within the scope of the present invention. Methods of solvation are generally known in the art.

### Methods of Preparation

Another aspect of the invention is a process for synthesizing any of the compounds described above or intermediates thereof, as described in the specification, in particular

The synthesis of epothilone A-derived oxazolines 4-6 in all cases is based on amino alcohol 3 as the central intermediate. As illustrated in Scheme 1, 3 is obtained through nucleophilic ring-opening of the epoxide moiety in epothilone A (1a) with azide anion (to produce 2) and subsequent reduction of the azide group under Staudinger conditions (Ph₃P/H₂O).

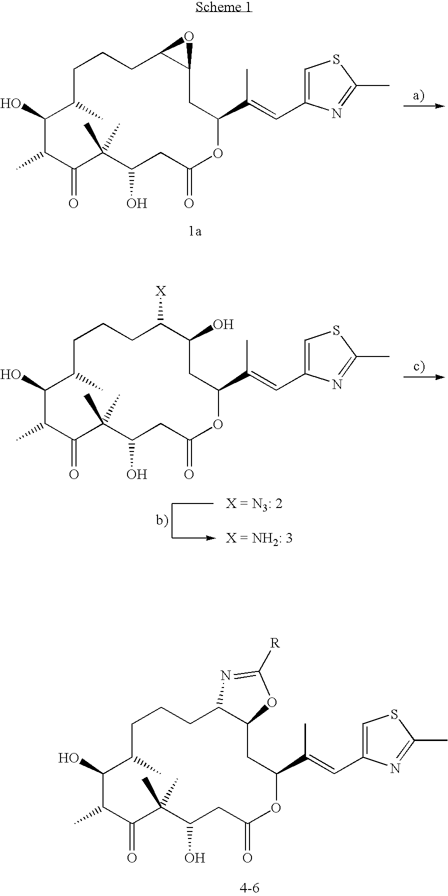

Scheme 1: a) LiN₃, NH₄Cl, DMF, 85° C., 24 h, 38%; b) Ph₃P, THF/H₂O 15/1, RT, 88 h, 50%; c) cf. text and Schemes 2, 3.

The structure of azido alcohol 2 (with a 12-azido group) as the major product of the reaction between epothilone A and LiN₃ in DMF in the presence of NH₄Cl (the conditions employed in this work) is firmly established by means of NMR spectroscopy; additional proof for this structural assignment comes from the subsequent X-ray crystal structure of amine 3 (as its hydroacetate), which showed the amino group to be attached to C-12 (and not C-13). The regiochemical course of the epoxide opening reaction is thus identical with that reported for the reaction of 12,13-bis-epi-epothilone A with NaN₃ in EtOH [1].

Our initial approach to the elaboration of amino alcohol 3 into the desired oxazolines involved reaction of this intermediate with the appropriate orthoester in refluxing EtOH. Thus, heating of 3 with the commercially available triethylorthoesters of acetic acid, propionic acid, valeric acid, 3-phenylsulfonyl-propionic acid, and benzoic acid or with tetraethylorthocarbonate produces analogs 4a, 4b, 4c, 4g, 5a, and 4e, respectively, in yields between 21%-74%. (For structures cf. Table 1). In general, 3 is employed in these reactions as the hydroacetate (which is directly obtained in the chromatographic purification process), except for the synthesis of 4a, where the reaction is conducted with the free amine in the presence of catalytic amounts of TFA. In addition to the intended oxazoline 4e, the reaction of the hydroacetate of 3 with tetraethylcarbonate also provides small quantities (8%) of oxazolidinone 4h, the formal hydrolysis product of 4e.

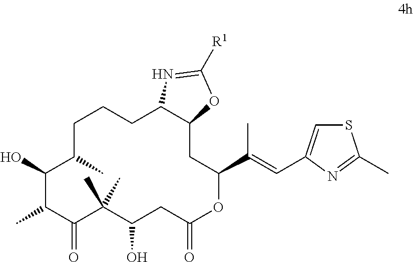

For the majority of target structures investigated the requisite orthoesters are not commercially available and are intended to be prepared from the corresponding nitrites through HCl-catalyzed iminoester formation and subsequent ethanolysis. This approach is followed in the synthesis of 4f, which is obtained from 3 in 55% yield, in spite of the fact that the starting orthoester can only be obtained in impure form. Subsequent experiments demonstrate that the formation of epothilone A-derived oxazolines in fact can also be achieved through direct reaction of 3 (as the hydroacetate) with crude iminoester hydrochlorides in refluxing DCE in the presence of catalytic amounts of ethanol (Scheme 2; for structures cf. Table).

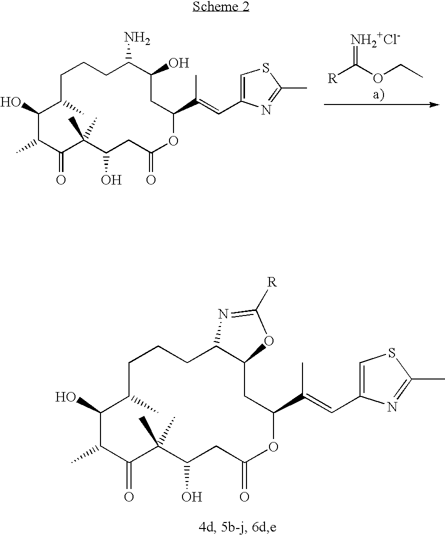

### Scheme 2: a) DCE, EtOH (cat.), 90° C., 2-24 h, 16-68%.

Using this procedure aryl-oxazolines 5b-j as well as the tert-butyl-substituted derivative 4d and pyridine-based analogs 6d and 6e are obtained in moderate to acceptable yields (16%-68%) without the need for prior orthoester preparation. For reasons that are not explored, however, this process generally proves to be less efficient in the preparation of pyridine-containing oxazolines 6. These analogs are largely prepared through treatment of the appropriate iminoester with Et₃N in EtOH at RT, addition of diethylether to this mixture and direct reaction of the precipitate forms with the hydroacetate of 3 in refluxing DCE. This process is summarized in Scheme 3 for 2,5-substituted pyridine derivatives 6f-j. Compounds 6a-c are obtained in an analogous way.

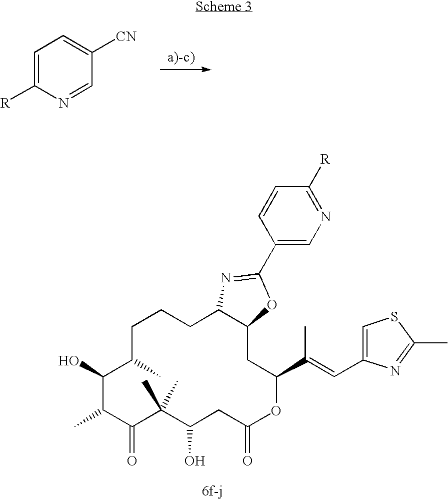

Scheme 1: a) HCl (gas), EtOH, 0° C.→RT, 16 h; b) Et₃N, EtOH, RT, 30 min-64 h; c) DCE, 90° C., 1-24 h, 21-57% (based on 3). HBr gas is used in step a) in the preparation of 6f.

It should be noted that the iminoesters obtained from cyano-pyridines with sat. HCl in EtOH do not produce clean NMR spectra (which may be attributable to the presence of differently protonated species) and that the products form upon treatment of these iminoesters with triethylamine (and which are probably mixtures of free iminoester and orthoester) are not characterized. In spite of the uncertainties associated with the purity and the exact nature of these precursors, however, the desired oxazolines can be obtained in acceptable yields and high purities. As 6-ethyl- and 6-iso-propyl-niconitile are not commercially available, these compounds are prepared from 6-chloro-3-cyano-pyridine by Fe(II)-catalyzed coupling with the corresponding Grignard reagents [2]. 6-tert-Butyl-3-cyano-pyridine is obtained from 3-cyano-pyridine and pivalic acid according to [3].
[1] A. Regueiro-Ren, R. M. Borzilleri, X. Zheng, S.-H. Kim, J. A. Johnson, C. R. Fairchild, F. Y. F. Lee, B. H. Long, G. D. Vite, Org. Lett. 2001, 3, 2693-2696.
[2] A. Fürstner, A. Leitner, M. Mendez, H. Krause, J. Am. Chem. Soc. 2002, 124, 13856-13863.
[3] A. Clerici, F. Minisci, O. Porta, Tetrahedron 1974, 30, 4201-4203.

### Abbreviations:

ACN, acetonitrile
DCE, dichloroethane (dried over molecular sieves)
DCM, dichloromethane
DEE, diethylether
FC, flash chromatography
NMP, N-methyl-pyrrolidone
RT, room temperature
TFA, trifluoroacetic acid

### HPLC Purification

Analytical HPLC is performed on a Waters Alliance system using a Waters Symmetry Shield® C₁₈ column. A gradient of ACN into water is employed over different time periods, depending on the specific separation problem. The solvents did not contain TFA. Purification by preparative HPLC is carried out on Gilson preparative HPLC systemt using a Waters Symmetry C18 column (5 mm) and the same solvent system as employed for analytical applications.

### Procedures for the Preparation of Individual Compounds

Azido alcohol 2: A solution of epothilone A (3.0 g; 6.08 mmole), LiN₃ (1.38 mg, 28.2 mmole), and NH₄Cl (354 mg, 6.6 mmole) in 8 ml of DMF is heated to 85° C. for 24 h. The reaction mixture is then concentrated, DCM (200 ml) is added, and the solution successively extracted with sat. aq. NaHCO₃ and water (100 ml each). It is then dried over MgSO₄ and the solvent is evaporated to yield 3.04 g of crude product. Purification of this material by FC in DCM/acetone/MeOH 87/10/3 gives 1.25 g (38%) of the target compound as an oil.

¹H-NMR (500 MHz, DMSO-d₆): δ=7.36 (s, 1H, H-19), 6.45 (s, 1H, H-17), 5.37 (d, 1H, H-15); 5.18 (d, 1H, 13-OH), 5.17 (d, 1H, 3-OH), 4.42 (m, 1H, H-3), 4.41 (d, 1H, 7-OH), 3.72 (m, 1H, H-13), 3.46 (m, t (br), 1H, H-7), 3.26 (m, 1H, H-12), 3.20 (q, 1H, H-6), 2.64 (s, 3H, H-21), 2.50 (m, 2H, H-2), 2.07 (s, 3H, H-27), 1.90 (m, 1H, H-14), 1.87 (m, 1H, H-11), 1.76 (m, 1H, H-11), 1.54 (m, 1H, H-11), 1.41 (m, 2H, H-10/H-9), 1.21 (m, 1H, H-10), 1.10 (m, 1H, H-8), 0.99 (m, 1H, H-9), 1.06 (s, 3H, H-22 or H-23), 1.05 (d, 3H, H-24), 0.87 (d, 3H, H-25), 0.85 (s, 3H, H-22 or H-23).

ESI-MS: 537.2 [M+H]⁺. HRMS: m/z 462.2259 [M+Na]⁺ calcd. for [C₂₆H₃₃NO₅+Na] 462.2256.

Amino alcohol 3: To a solution of 1.16 g (2.16 mmole) of azido alcohol 2 in a mixture of 38 ml of THF and 2.4 ml of water are added 1.132 g Ph₃P (4.32 mmole). After stirring at RT for 88 h the mixture is concentrated and directly submitted to FC in CHCl₃/MeOH/H₂O/AcOH 75/27/3/0.5 to provide the target product as the corresponding hydroacetate (546 mg, 50%) in oily form. Lyophilization of the material from water/CH₃CN 3/1 gives the hydroacetate of 3 as a fluffy white powder.

¹H-NMR (500 MHz, DMSO-d₃): δ=7.35 (s, 1H, H-19), 6.43 (s, 1H, H-17), 5.36 (m, 1H, H-15), 4.47 (t, 1H, H-3), ca. 3.5 (broad signal overlapping with signals for H-6, H-7, H-12, and H-13), 3.20 (m, 1H), 2.70 (m, 1H), 2.61 (s, 3H, H-21), 2.04 (s, 3H, H-27), 1.80 (s, 3H, AcOH), 1.75 (m, 2H, H-11/H-14), 1.55 (m, 1H, H-14), 1.35 (m, 3H, H-11/H-10/H-9), 1.10 (m, 2H, H-10/H-8), 1.08 (d, 3H), 1.05 (s, 3H, H-22 or H-23), 0.90 (m, 1H, H-9), 0.85 (d, 3H), 0.82 (s, 3H, H-22 or H-23).

HPLC purity >98%. ESI-MS: 511.0 [M+H]⁺.

In other experiments 3 is purified by preparative HPLC without prior FC(H₂O/CH₃CN without inclusion of TFA). Through this procedure the compound is obtained as the free amine, which is used in the preparation of oxazoline 4a.

4a: Amino alcohol 3 (free base; 10.2 mg, 0.02 mmole) is dissolved in a solution of 9.7 mg (0.06 mmole) of triethyl orthoacetate and 1.37 mg (0.012 mmole) of TFA in 200 μL of DCE (taken from a stock solution of 275 μL of triethyl orthoacetate and 34.5 μL of TFA in 5 ml of DCM) and the mixture is heated to 90° C. for 3 h. It is then diluted with 1 ml of ACN and directly purified by preparative HPLC (5%→100% ACN in 100 min) to provide 6.1 mg of material that is repurified by FC in 5% MeOH/DCM→10% MeOH/DCM. This procedure gives 2.45 mg of the target compound 4a (23%)→0.35 mg (3%) of hydrolyzed material (N-acetyl-3; ESI-MS: 553.3 [M+H]⁺).

¹H-NMR (500 MHz, DMSO-d₆): δ=7.36 (s, 1H, H-19), 6.51 (s, 1H, H-17), 5.30 (d, 1H, H-15), 5.12 (d, 1H, 3-OH), 4.28 (m, 2H, H-3+7-OH), 4.15 (m, 1H, H-13), 3.54 (m, 1H, H-12), 3.47 (m, 1H, H-7), 3.17 (m, 1H, H-6), 2.65 (s, 3H, H-21), 2.41 (dd, 1H, H-2), 2.29 (dd, 1H, H-2), 2.09 (s, 3H, H-27), 2.08 (m, 1H), 1.90 (m, 2H), 1.85 (s, 3H, CH₃ oxazoline), 1.65 (m, 1H), 1.55 (m, 1H), 1.35 (m, 2H), 1.23 (s, 3H), 1.15 (m, 1H), 1.05 (m, 1H), 0.94 (d, 3H), 0.88 (s, 3H), 0.84 (d, 3H).

ESI-MS: 535.3 [M+H]⁺.

4b: Amino alcohol 3 (hydroacetate; 51 mg, 0.1 mmole) and 40.2 mg (0.3 mmole) of triethyl orthopropionate are dissolved in 600 μL of dry DCE and the mixture is heated to 90° C. for 2 h. After cooling to RT the reaction mixture is directly submitted to FC in 4% MeOH/DCM to yield 33.6 mg of the title compound (61%). Dissolution in ACN/water 2/1 and lyophilization gives the product as a fluffy white powder.

¹H-NMR (500 MHz, DMSO-d₆): δ=7.36 (s, 1H, H-19), 6.51 (s, 1H, H-17), 5.27 (d, 1H, H-15), 5.11 (d, 1H, 3-OH), 4.29 (m, 2H, H-13+7-OH), 4.17 (m, 1H, H-3), 3.54 (m, 1H, H-12), 3.49 (m, 1H, H-7), 3.19 (m, 1H, H-6), 2.65 (s, 3H, H-21), 2.42 (dd, 1H, H-2), 2.29 (dd, 1H, H-2), 2.18 (m, 2H, Et oxazoline), 2.08 (s, 3H, H-27), 1.88 (d, 1H, H-14), 1.83 (d, 1H, H-14), 1.68 (m, 1H, H-11), 1.55 (m, 1H), 1.35 (m, 2H, H-9), 1.23 (s, 3H, H22 or H-23, +m, 1H), 1.15 (m, 1H), 1.14 (m, 1H, H-11), 1.05 (t, 3H, Et oxazoline, +m, 1H), 0.94 (d, 3H, H-24), 0.88 (s, 3H, H-22 or H-23), 0.84 (d, 3H, H-25).

ESI-MS: 549 [M+H]⁺; 571.4 [M+Na]⁺. HRMS: m/z 571.2814 [M+Na]⁺ calcd. for [C₂₉H₄₄N₂O₆S+Na] 571.2818.

4c: To a solution of amino alcohol 3 (hydroacetate; 51 mg, 0.1 mmole) in 600 μL of DCE are added 48.6 mg (0.3 mmole) of trimethyl orthovalerate and the mixture is heated to 90° C. for 3 h. After cooling to RT the reaction mixture is directly submitted to FC in 3% MeOH/DCM to yield 36.3 mg (63%) of the title compound. Dissolution of the material in ACN/water 2/1 and lyophilization gives the product as a fluffy white powder.

¹H-NMR (500 MHz, DMSO-d₆): δ=7.36 (s, 1H, H-19), 6.50 (s, 1H, H-17), 5.26 (d, 1H, H-15), 5.12 (d, 1H, 3-OH), 4.29 (m, 2H, H-13+7-OH), 4.16 (m, 1H, H-3), 3.55 (m, 1H, H-12), 3.48 (m, 1H, H-7), 3.19 (m, 1H, H-6), 2.64 (s, 3H, H-21), 2.43 (dd, 1H, H-2), 2.30 (dd, 1H, H-2), 2.17 (t, 2H, Bu oxazoline), 2.08 (s, 3H, H-27), 1.87 (dd, 1H, H-14), 1.81 (dd, 1H, H-14), 1.65 (m, 1H), 1.50 (m, 3H), 1.40-1.20 (m, 5H), 1.23 (s, 3H, H22 or H-23, +m, 1H), 1.15 (m, 1H), 1.05 (m, 1H), 1.05 (m, 1H), 0.93 (d, 3H, H-24), 0.88 (s, 3H, H-22 or H-23), 0.86 (t, 3H, Bu oxazoline), 0.83 (d, 3H, H-25).

ESI-MS: 577.4 [M+H]⁺; 599.5 [M+Na]⁺. HRMS: m/z 577.3311 [M+H]⁺ calcd. for [C₃₁H₄₈N₂O₆S+H] 577.3309.

4d: A. Preparation of the imino ester: A mixture of 8.3+g-(0.1 mole) of trimethyl acetonitrile and 5.05 g of EtOH (0.11 mole) is treated with HCl gas at 5° C. until saturation. The mixture is then stored at the same temperature for 18 h and 20 ml of DEE are added. The resulting precipitate is collected by filtration, ished with DEE and dried to yield 3.9 g (24%) of the crude imino ester hydrochloride as a white solid. (¹H-NMR (400 MHz, DMSO-d₆): δ=4.42 (q, 2H), 1.35 (t, 3H), 1.25 (s, 9H)).

B. Oxazoline formation: To a solution of amino alcohol 3 (hydroacetate; 25.5 mg, 0.05 mmole) in 300 μL of DCE are added 18 mg (0.11 mmole) of the above imino ester hydrochloride and the mixture is heated to 90° C. for 2 h. After that time 20 μL of EtOH are added and heating at 90° C. is continued for 4 h, when additional 50 μL of EtOH are added. Heating is continued for further 18 h and the reaction mixture is directly submitted to FC in 3% MeOH/DCM to yield 4.5 mg (16%) of the title compound 4d. Dissolution of the material in ACN/water 2/1 and lyophilization gives the product as a fluffy white powder.

¹H-NMR (500 MHz, DMSO-d₆): δ=7.35 (s, 1H, H-19), 6.51 (s, 1H, H-17), 5.26 (d, 1H, H-15), 5.15 (d, 1H, 3-OH), 4.32 (m, 2H, H-13+7-OH), 4.24 (m, 1H, H-3), 3.51 (m, 2H, H-12+H-7), 3.21 (m, 1H, H-6), 2.64 (s, 3H, H-21), 2.44 (dd, 1H, H-2, overlapping with solvent signal), 2.31 (dd, 1H, H-2), 2.07 (s, 3H, H-27), 1.88 (dd, 1H, H-14), 1.77 (dd, 1H, H-14), 1.70 (m, 1H), 1.51 (m, 1H), 1.36 (m, 2H), 1.22 (s, 3H, H22 or H-23), 1.13 (s, 9H), signals at 1.22 and 1.13 overlapping m, 3H, 0.94 (d, 3H, H-24), 0.88 (s, 3H, H-22 or H-23), 0.84 (d, 3H, H-25).

ESI-MS: 577.0 [M+H]⁺.

4e and 4h: To a solution of amino alcohol 3 (hydroacetate; 51 mg, 0.1 mmole) in 600 μL of DCE are added 40.8 mg (0.3 mmole) of tetramethyl orthocarbonate and the mixture is heated to 90° C. for 3 h. After cooling to RT the reaction mixture is directly submitted to FC in 4% MeOH/DCM to yield 11.5 mg of 4e (21%) and 4.3 mg (8%) of 4h. Dissolution of these materials in ACN/water 2/1 and lyophilization gives the products as fluffy white powders.

4e: ¹H-NMR (500 MHz, DMSO-d₆): δ=7.36 (s, 1H, H-19), 6.51 (s, 1H, H-17), 5.26 (d, 1H, H-15), 5.12 (d, 1H, 3-OH), 4.50 (t, 1H, H-13), 4.28 (d, 1H, 7-OH), 4.13 (m, 1H, H-3), 3.47 (m, 1H, H-7), 3.41 (m, 1H, H-12), 3.16 (m, 1H, H-6), 2.65 (s, 3H, H-21), 2.44 (dd, 1H, H-2), 2.30 (dd, 1H, H-2), 2.10 (s, 3H, H-27), 1.97 (dd, 2H, H-14), 1.55 (m, 2H), 1.50 (m, 3H), 1.40 (m, 1H), 1.32 (m, 2H), 1.25 (s, 3H, H22 or H-23, +m, 1H), 1.15 (m, 1H), 1.05 (m, 1+H), 0.95 (d, 3H, H-24), 0.88 (s, 3H, H-22 or H-23), 0.85 (d, 3H, H-25).

ESI-MS: 551.3 [M+H]⁺; 573.3 [M+Na]⁺. HRMS: m/z 573.2610 [M+Na]⁺ calcd. for [C₂₇H₄₀N₂O₇S+Na] 573.2613.

4h: ¹H-NMR (500 MHz, DMSO-d₆): δ=7.63 (s, 1H, NH), 7.37 (s, 1H, H-19), 6.51 (s, 1H, H-17), 5.29 (dd, 1H, H-15), 5.12 (d, 1H, 3-OH), 4.37 (q, 1H, H-13), 4.31 (d, 1H, 7-OH), 4.09 (t (br), 1H, H-3), 3.75 (s, 3H, OCH₃), 3.54 (m, 1H, H-13), 3.49 (m, 1H, H-7), 3.16 (m, 1H, H-6), 2.65 (s, 3H, H-21), 2.42 (dd, 1H, H-2), 2.29 (dd, 1H, H-2), 2.09 (s, 3H, H-27), 1.94 (m, 2H, H-14), 1.62 (m, 1H), 1.53 (m, 1H), 1.35 (m, 3H), 1.24 (s, 3H, H22 or H-23, +m, 1H), 1.15 (m, 1H), 1.05 (m, 1H), 0.94 (d, 3H, H-24), 0.88 (s, 3H, H-22 or H-23), 0.84 (d, 3H, H-25).

ESI-MS: 537.3 [M+H]⁺; 559.3 [M+Na]⁺. HRMS: m/z 559.2454 [M+Na]⁺ calcd. for [C₂₇H₄₀N₂O₇S+Na] 559.2463.

4f: A. Preparation of the orthoester: A mixture of 23.4 of benzyl cyanide (0.2 mole) and 10.1 g of dry EtOH (0.22 mole) is treated with HCl gas at 5° C. until saturation. The mixture is then stored at the same temperature for 18 h and 40 ml of DEE are added. The resulting precipitate is collected by filtration, ished with DEE and dried to yield 31.3 g (79%) of the crude imino ester hydrochloride as a white solid. This material is dissolved in 50 ml of EtOH and the mixture is kept at RT for 2 d. DEE (25 ml) is then added and NH₄Cl is removed by filtration. The filtrate is evaporated in vacuo at RT to produce a yellow oil containing a precipitate of phenylacetamide. The solid material is removed by filtration and the filtrate subjected to Kugelrohr distillation to provide 23.9 g (64%) of triethyl-orthophenylacetate as a colorless oil (3 runs, bp. 90° C./0.1 mbar). According to NMR this material is still impure, but is used in the next step without further purification.

B. Oxazoline formation: To a solution of amino alcohol 3 (hydroacetate; 51 mg, 0.1 mmole) in 600 μL of DCE are added 71.4 mg (0.3 mmole) of the above crude ortho ester and the mixture is heated to 90° C. for 3 h. After cooling to RT the reaction mixture is directly submitted to FC in 3% MeOH/DCM to yield 48 mg of impure product. This material is re-chromatographed in 2% MeOH/DCM to provide 33.6 mg (55%) of the title compound 4f. Dissolution of the material in ACN/water 2/1 and lyophilization gives the product as a fluffy white powder.

¹H-NMR (500 MHz, DMSO-d₆): δ=7.34 (s, 1H, H-19), 7.32-7.20 (m, 5H), 6.47 (s, 1H, H-17), 5.19 (d, 1H, H-15), 5.12 (d, 1H, 3-OH), 4.34 (t, 1H, H-13), 4.30 (d, 1H, 7-OH), 4.15 (m, 1H, H-3), 3.59 (m, 1H, H-12), 3.56 (d, 2H, Bn oxazoline), 3.48 (m, 1H, H-7), 3.16 (m, 1H, H-6), 2.64 (s, 3H, H-21), 2.41 (dd, 1H, H-2), 2.28 (dd, 1H, H-2), 2.03 (s, 3H, H-27), 1.87 (dd, 1H, H-14), 1.78 (dd, 1H, H-14), 1.68 (m, 1H), 1.50 (m, 1H), 1.33 (m, 2H), 1.23 (s, 3H, H22 or H-23, +m, 1H), 1.15 (m, 1H), 1.05 (m, 1H), 0.93 (d, 3H, H-24), 0.86 (s, 3H, H-22 or H-23), 0.83 (d, 3H, H-25).

ESI-MS: 610.9.3 [M+H]⁺.

4g: To a solution of amino alcohol 3 (hydroacetate; 51 mg, 0.1 mmole) in 600 μL of DCE is added a solution of 60 mg (0.3 mmole) of triethyl-3-phenylsulfonyl-orthopropionate in 600 μL of DCE. and the mixture is heated to 80° C. for 1 h and then to 90° C. for 3 h. The reaction mixture is directly submitted to FC in 4% MeOH/DCM to yield 34.5 mg (50%) of the title compound 4g. Dissolution in ACN/water 2/1 and lyophilization gives the product as a fluffy white powder.

¹H-NMR (500 MHz, DMSO-d₆): δ=7.90 (d, 2H), 7.76 (m, 1H), 7.65 (m, 2H), 7.32 (s, 1H, H-19), 6.50 (s, 1H, H-17), 5.23 (d, 1H, H-15), 5.10 (d, 1H, 3-OH), 4.27 (d, 1H, 7-OH), 4.23 (t, 1H, H-13), 4.14 (m, 1H, H-3), 3.57 (t, 2H, —CH₂SO₂—), 3.47 (m, 2H, H-12+H-7), 3.18 (m, 1H, H-6), 2.65 (s, 3H, H-21), 2.41 (dd, 1H, H-2), 2.29 (dd, 1H, H-2), 2.07 (s, 3H, H-27), 1.79 (m, 2H, H-14), 1.60 (m, 1H, H-11), 1.52 (m, 1H, H-8), 1.36 (m, 2H, H-9), 1.23 (s, 3H, H22 or H-23, +m (1.20-1.00), 1H, H-11), 0.92 (d, 3H, H-24), 0.87 (s, 3H, H-22 or H-23), 0.84 (d, 3H, H-25).

ESI-MS: 688.8 [M+H]⁺. HRMS: m/z 711.2750 [M+Na]⁺ calcd. for [C₃₅H₄₈N₂O₈S₃+Na] 711.2750.

5a: Amino alcohol 3 (hydroacetate; 51 mg, 0.100 mmole) is dissolved in a solution of 68 μL (0.300 mmole) of triethyl orthobenzoate in 600 μL of dry DCE and the mixture is heated to 90° C. for 2 h. After cooling to RT, the reaction mixture is directly submitted to FC in 3% MeOH/DCM to yield 44.0 mg (74%) of the title compound 5a. Dissolution in ACN/water 2/1 and lyophilization gives the product as a fluffy white powder.

¹H-NMR (500 MHz, DMSO-d₆): δ=7.85 (d, 2H), 7.54 (m, 1H), 7.47 (m, 2H), 7.36 (s, 1H, H-19), 6.56 (s, 1H, H-17), 5.43 (dd, 1H, H-15), 5.17 (d, 1H, 3-OH), 4.57 (t, 1H, H-13), 4.33 (d, 1H, 7-OH), 4.20 (m, 1H, H-3), 3.83 (m, 1H, H-12), 3.52 (m, 1H, H-7), 3.21 (m, 1H, H-6), 2.64 (s, 3H, H-21), 2.46 (dd, 1H, H-2), 2.33 (dd, 1H, H-2), 2.11 (s, 3H, H-27), 1.99 (m, 2H, 1H-14), 1.80 (m, 1H, H-11), 1.53 (m, 1H, H-8), 1.38 (m, 3H), 1.25 (s, 3H, H22 or H-23), 1.22 (m, 1H), 0.95 (d, 3H, H-24), 0.89 (s, 3H, H-22 or H-23), 0.84 (d; 3H, H-25).

ESI-MS: 597.3 [M+H]⁺. HRMS: m/z 619.2812 [M+Na]⁺ calcd. for [C₃₃H₄₄N₂O₆S+Na] 619.2818.

5b: A. Preparation of the iminoester hydrochloride: A solution of 1.0 g (7.5 mmole) of 4-fluoro benzonitrile in 8 ml EtOH/10 ml DEE is treated with HCl gas at 0° C. until saturation and the solution is kept at RT over night. It is then evaporated to dryness and the residue dried in vacuo to provide 1.60 g the crude imino ester hydrochloride. (¹H-NMR (500 MHz, DMSO-d₆): δ=11.90 (s (br), 1H), 8.23 (m, 2H), 7.50 (t, 2H), 4.60 (q, 2H), 1.48 (t, 3H)).

B. Oxazoline formation: 30.5 mg (0.150 mmole) of the above iminoester hydrochloride are added to a solution of amino alcohol 3 (hydroacetate; 25.5 mg, 0.050 mmole) in 300 μL of DCE together with 20 μL of EtOH and the mixture is heated to 90° C. for 5 h. After cooling to RT, the reaction mixture is directly submitted to FC in 4% MeOH/DCM to yield 19.1 mg (62%) of pure title compound 5b.

¹H-NMR (500 MHz, DMSO-d₆): δ=7.91 (m, 2H), 7.36 (s, 1H, H-19), 7.30 (t, 2H), 6.56 (s, 1H, H-17), 5.44 (dd, 1H, H-15), 5.16 (d, 1H, 3-OH), 4.58 (m, 1H, H-13), 4.29 (d, 1H, 7-OH), 4.20 (m, 1H, H-3), 3.83 (m, 1H, H-12), 3.51 (m, 1H, H-7), 3.21 (m, 1H, H-6), 2.64 (s, 3H, H-21), 2.46 (dd, 1H, H-2), 2.33 (dd, 1H, H-2), 2.11 (s, 3H, H-27), 1.99 (m, 2H, H-14), 1.68 (m, 1H), 1.52 (m, 1H), 1.38 (m, 3H), 1.25 (s, 3H, H-22 or H-23+m, 1H), 1.11 (m, 1H), 0.95 (d, 3H, H-24), 0.89 (s, 3H, H-22 or H-23), 0.84 (d, 3H, H-25).

ESI-MS: 614.9 [M+H]⁺. HRMS: m/z 637.2724 [M+Na]⁺ calcd. for [C₃₃H₄₃FN₂O₆S+Na] 637.2728.

5c: To a solution of amino alcohol 3 (hydroacetate; 25.5 mg, 0.050 mmole) in 300 μL of DCE are added 33.0 mg (0.150 mmole) of ethyl p-chlorobenzimidate hydrochloride and 20 μL of EtOH and the mixture is heated to 90° C. for 5 h. After cooling to RT the reaction mixture is then directly subjected to FC in 4% MeOH/DCM to yield 10.6 mg (34%) of the title compound 5c.

¹H-NMR (500 MHz, DMSO-d₆): δ=7.85 (d, 2H), 7.55 (d, 2H), 7.36 (s, 1H, H-19), 6.56 (s, 1H, H-17), 5.43 (t (br), 1H, H-15), 5.16 (d, 1H, 3-OH), 4.59 (m, 1H, H-13), 4.31 (d, 1H, 7-OH), 4.19 (m, 1H, H-3), 3.85 (m, 1H, H-12), 3.50 (m, 1H, H-7), 3.20 (m, 1H, H-6), 2.64 (s, 3H, H-21), 2.45 (dd, 1H, H-2), 2.33 (dd, 1H, H-2), 2.11 (s, 3H H-27), 1.99 (m, 2H, H-14), 1.80 (m, 1H), 1.54 (m, 1H), 1.40 (m, 3H), 1.25 (s, 3H, H-22 or H-23+m, 1H), 1.13 (m, 1H), 0.95 (d, 3H, H-24), 0.89 (s, 3H, H-22 or H-23), 0.84 (d, 3H, H-25).

ESI-MS: 630.8, 100%, 632.8, 45% [M+H]⁺. HRMS (monoisotopic): m/z 653.2424 [M+Na]+calcd. for [C₃₃H₄₃ClN₂O₆S+Na] 637.2728.

5d: A. Preparation of the iminoester hydrochloride: A solution of 1.0 g (5.5 mmole) of 4 bromobenzonitrile in a mixture of 8 ml of EtOH and 10 ml of DEE is saturated with HCl gas at 0° C. After storage of the mixture at RT over night evaporation of the solvents provides 1.40 g of the iminoester hydrochloride as a white solid. (ESI-MS: 228, 100%, 230, 80% [M+H]⁺. ¹H-NMR (400 MHz, DMSO-d₆): δ=8.05 (d, 2H), 7.85 (d, 2H), 4.60 (q, 2H), 1.45 (t, 3H)).

B. Oxazoline formation: To a solution of amino alcohol 3 (hydroacetate; 20.4 mg, 0.04 mmole) in 300 μL of DCE are added 32.0 mg (0.150 mmole) of KH-2495 and 20 μL of EtOH and the mixture is heated to 90° C. for 3 h. After cooling to RT the reaction mixture is directly submitted to FC in 3% MeOH/DCM to yield 8.1 mg of slightly impure product which is purified by preparative HPLC (30%→100% ACN in 100 min) to yield 6.33 mg (24%) of the title compound 5d.

¹H-NMR (500 MHz, DMSO-d₆): δ=7.78 (d, 2H), 7.69 (d, 2H), 7.36 (s, 1H, H-19), 6.56 (s, 1H, H-17), 5.43 (m, 1H, H-15), 5.17 (s (br), 1H, 3-OH), 4.58 (m, 1H, H-13), 4.33 (d, 1H, 7-OH), 4.19 (d (br), 1H, H-3), 3.83 (m, 1H, H-12), 3.50 (m, 1H, H-7), 3.20 (m, 1H, H-6), 2.64 (s, 3H, H-21), 2.45 (dd, 1H, H-2), 2.32 (dd, 1H, H-2), 2.11 (s, 3H, H-27), 1.99 (m, 2H, H-14), 1.78 (m, 1H), 1.51 (m, 1H), 1.38 (m, 3H), 1.25 (s, 3H, H-22 or H-23+m, 1H), 1.10 (m, 1H), 0.94 (d, 3H, H-24), 0.89 (s, 3H, H-22 or H-23), 0.84 (d, 3H, H-25).

ESI-MS: 674.9, 100%, 676.9, 95% [M+H]⁺. HRMS (monoisotopic): m/z 675.2104 [M+Na]⁺ calcd. for [C₃₃H₄₃BrN₂O₆S+Na] 675.2104.

5e: A. Preparation of the iminoester hydrochloride: A solution of 5.0 g (42.7 mmole) of p-tolunitrile in 40 ml EtOH/50 ml DEE is treated with HCl gas at 0° C. until saturation. The solution is then kept at RT over night, the solvent removed by evaporation and the residue dried in vacuo to provide 8.81 g of the crude iminoester hydrochloride as a white solid. (¹H-NMR (400 MHz, DMSO-d₆): δ=8.04 (d, 2H), 7.44 (d, 2H), 4.62 (q, 2H), 2.40 (s, 3H), 1.47 (t, 3H)).

B. Oxazoline formation: 30.0 mg (0.150 mmole) of the above iminoester hydrochloride are added to a solution of amino alcohol 3 (hydroacetate; 25.5 mg, 0.050 mmole) in 300 μL of DCE together with 20 μL of EtOH and the mixture is heated to 90° C. for 3 h. After cooling to RT the reaction mixture is directly submitted to FC in 2% MeOH/DCM to yield 12.4 mg (41%) of the title compound 5e. Dissolution of the material in ACN/water 2/1 and lyophilization gives the product as a fluffy white powder.

¹H-NMR (500 MHz, DMSO-d₆): δ=7.74 (d, 2H), 7.36 (s, 1H, H-19), 7.28 (d, 2H), 6.56 (s, 1H, H-17), 5.41 (dd (br), 1H, H-15), 5.16 (d, 1H, 3-OH), 4.54 (m, 1H, H-13), 4.32 (d, 1H, 7-OH), 4.21 (m (br), 1H, H-3), 3.81 (m, 1H, H-12), 3.52 (m, 1H, H-7), 3.20 (m, 1H, H-6), 2.64 (s, 3H, H-21), 2.46 (dd, 1H, H-2), 2.32 (dd, 1H, H-2), 2.11 (s, 3H, H-27), 1.98 (m, 2H, H-14), 1.70 (m, 1H), 1.53 (m, 1H), 1.37 (m, 3H), 1.24 (s, 3H, H-22 or H-23+m, 1H), 1.12 (m, 1H), 0.95 (d, 3H, H-24), 0.89 (s, 3H, H-22 or H-23), 0.84 (d, 3H, H-25).

ESI-MS: 611.2, 100% [M+H]⁺; 633.2, 23% [M+Na]⁺. HRMS: m/z 611.3155 [M+H]⁺ calcd. for [C₃₄H₄₆N₂O₆S+H] 611.3161.

5f: A. Preparation of the iminoester hydrochloride: A solution of 1.0 g (5.85 mmole) of trifluoro-p-tolunitrile in 8 ml EtOH/10 ml DEE is treated with HCl gas at 0° C. until saturation. The solution is then kept at RT over night, the solvent removed by evaporation and the residue dried in vacuo to provide 1.44 g of the imino ester. (¹H-NMR (400 MHz, DMSO-d₆): δ=8.29 (d, 2H), 8.04 (d, 2H), 4.65 (q, 2H), 1.48 (t, 3H)).

B. Oxazoline formation: 38.0 mg (0.15 mmole) of the above iminoester hydrochloride are added to a solution of amino alcohol 3 (hydroacetate; 25.5 mg, 0.05 mmole) in 300 μL of DCE together with 20 μL of EtOH and the mixture is heated to 90° C. for 3 h. After cooling to RT the reaction mixture is directly submitted to FC in 3% MeOH/DCM to yield 16.8 mg of impure material. Re-chromatography in 3% MeOH/DCM gives 5.6 mg of pure title compound. Impure fractions are combined (11.2 mg) and purified by preparative HPLC (30%→100% ACN in 100 min) to provide additional 8.55 mg of pure lyophilized 5f. Total yield: 14.15 mg (43%).

¹H-NMR (500 MHz, DMSO-d₆): δ=8.08 (d, 2H), 7.88 (d, 2H), 7.36 (s, 1H, H-19), 6.59 (s, 1H, H-17), 5.47 (t (br), 1H, H-15), 5.17 (d, 1H, 3-OH), 4.66 (m, 1H, H-13), 4.31 (d, 1H, 7-OH), 4.21 (m, 1H, H-3), 3.92 (m, 1H, H-12), 3.53 (m, 1H, H-7), 3.21 (m, 1H, H-6), 2.67 (s, 3H, H-21), 2.48 (dd, 1H, H-2), 2.36 (dd, 1H, H-2), 2.13 (s, 3H, H-27), 2.04 (m, 2H, H-14), 1.70 (m, 1H), 1.53 (m, 1H), 1.43 (m, 3H), 1.24 (s, 3H, H-22 or H-23), 1.15 (m, 1H), 0.95 (d, 3H, H-24+m, 1H), 0.92 (s, 3H, H-22 or H-23), 0.86 (d, 3H, H-25).

ESI-MS: 665.1 [M+H]⁺. HRMS: m/z 687.2692 [M+Na]⁺ calcd. for [C₃₄H₄₃F₃N₂O₆S+Na] 687.2689.

5g: A. Preparation of the iminoester hydrochloride: A solution of 1.0 g (8.4 mmole) of 4-hydroxybenzonitrile in a mixture of 8 ml EtOH and 10 ml of DEE is saturated with HCl gas at 0° C. After over night storage at RT 50 ml of DEE are added and the crude imino ester hydrochloride is isolated by filtration (1.72 g; ¹H-NMR (400 MHz, DMSO-d₆): δ=8.05 (d, 2H), 7.00 (d, 2H), 4.56 (q, 2H), 1.45 (t, 3H)).

B. Oxazoline formation: To a solution of amino alcohol 3 (hydroacetate; 25.5 mg, 0.050 mmole) in 300 μL of DCE are added 33.0 mg (0.150 mmole) of the above iminoester hydrochloride and 20 μL of EtOH and the mixture is heated to 90° C. for 3 h. After cooling to RT the reaction mixture is directly subjected to FC in 5% MeOH/DCM to yield 12.54 mg (41%) of the title compound 5g.

¹H-NMR (500 MHz, DMSO-d₆): δ=10.08 (s (br), 1H, Ar—OH); 7.70 (d, 2H), 7.38 (s, 1H, H-19), 6.83 (d, 2H), 6.57 (s, 1H, H-17), 5.43 (d (br), 1H, H-15), 5.18 (d, 1H, 3-OH), 4.52 (t (br), 1H, H-13), 4.33 (d, 1H, 7-OH), 4.24 (m, 1H, H-3), 3.78 (m, 1H, H-12), 3.54 (m, 1H, H-7), 3.23 (m, 1H, H-6), 2.67 (s, 3H, H-21), 2.50 (dd, 1H, H-2), 2.34 (dd, 1H, H-2), 2.13 (s, 3H, H-27), 1.99 (m, 2H, H-14), 1.80 (m, 1H), 1.56 (m, 1H), 1.39 (m, 3H), 1.27 (s, 3H, H-22 or H-23+m, 1H), 1.15 (m, 1H), 0.96 (d, 3H, H-24), 0.92 (s, 3H, H-22 or H-23), 0.87 (d, 3H, H-25).

ESI-MS: 613.2 [M+H]⁺. HRMS: m/z 613.2947 [M+H]⁺ calcd. for [C₃₃H₄₄N₂O₆S+H] 613.2948.

5h: A. Preparation of the iminoester hydrochloride: A solution of 1.0 g (7.5 mmole) of 4-methoxy benzonitrile in 8 ml EtOH/10 ml DEE is treated with HCl gas at 0° C. until saturation. The solution is then kept at RT over night, the solvent removed by evaporation and the residue dried in vacuo to provide 1.65 g of the crude imino ester hydrochloride. (¹H-NMR (400 MHz, DMSO-d₆): δ=8.15 (d, 2H), 7.17 (d, 2H), 4.58 (q, 2H), 3.90 (s, 3H), 1.45 (t, 3H)).

B. Oxazoline formation: To a solution of amino alcohol 3 (hydroacetate; 25.5 mg, 0.050 mmole) in 300 μL of DCE are added 32.0 mg (0.150 mmole) of the above iminoester hydrochloride and 20 μL of EtOH and the mixture is heated to 90° C. for 3 h. After cooling to RT the reaction mixture is directly submitted to FC in 5% MeOH/DCM to yield to yield: 16.5 mg of slightly impure material, which is further purified by preparative HPLC (30%→100% ACN in 100 min) to provide 11.85 mg (38%) of pure title compound 5h.

¹H-NMR (500 MHz, DMSO-d₆): δ=7.78 (d, 2H), 7.36 (s, 1H, H-19), 7.01 (d, 2H), 6.56 (s, 1H, H-17), 5.43 (d (br), 1H, H-15), 5.16 (d, 1H, 3-OH), 4.52 (t (br), 1H, H-13), 4.30 (d, 1H, 7-OH), 4.21 (m, 1H, H-3), 3.80 (s, 3H), 3.78 (m, 1H, H-12), 3.51 (m, 1H, H-7), 3.21 (m, 1H, H-6), 2.67 (s, 3H, H-21), 2.45 (dd, 1H, H-2), 2.33 (dd, 1H, H-2), 2.11 (s, 3H, H-27), 1.97 (m, 2H, H-14), 1.70 (m, 1H), 1.55 (m, 1H), 1.40 (m, 3H), 1.24 (s, 3H, H-22 or H-23+m, 1H), 1.15 (m, 1H), 0.95 (d, 3H, H-24), 0.89 (s, 3H, H-22 or H-23), 0.84 (d, 3H, H-25).

ESI-MS: 627.3 [M+H]⁺. HRMS: m/z 627.3104 [M+Na]⁺ calcd. for [C₃₄H₄₆N₂O₇S+Na] 627.3102.

5i: A. Preparation of the iminoester hydrochloride: A solution of 5.0 g (36.3 mmole) of 3-chloro-benzonitrile in 40 ml EtOH/50 ml DEE is treated with HCl gas at 0° C. until saturation. The solution is then kept at RT over night, the solvent removed by evaporation and the residue dried in vacuo to provide 7.70 g of the crude iminoester hydrochloride. (¹H-NMR (400 MHz, DMSO-d₆): δ=8.18 (d, 2H), 8.05 (dd, 1H), 7.86 (t, 1H), 4.60 (q, 2H), 1.49 (t, 3H)).

B. Oxazoline formation: To a solution of amino alcohol 3 (hydroacetate; 25.5 mg, 0.050 mmole) in 300 μL of DCE are added 33.0 mg (0.150 mmole) of the above iminoester hydrochloride and 20 μL of EtOH and the mixture is heated to 90° C. for 2 h. After cooling to RT the reaction mixture is directly submitted to FC in 3% MeOH/DCM to yield 18.5 mg (59%) of the title compound 51. Dissolution of the material in ACN/water 2/1 and lyophilization gives the product as a fluffy white powder.

¹H-NMR (500 MHz, DMSO-d₆): δ=7.82 (m, 2H), 7.61 (dd, 1H), 7.51 (t, 1H), 7.36 (s, 1H, H-19), 6.57 (s, 1H, H-17), 5.45 (t, 1H, H-15), 5.15 (d, 1H, 3-OH), 4.60 (m, 1H, H-13), 4.28 (d, 1H, 7-OH), 4.19 (m, 1H, H-3), 3.86 (m, 1H, H-12), 3.51 (m, 1H, H-7), 3.20 (m, 1H, H-6), 2.65 (s, 3H, H-21), 2.45 (dd, 1H, H-2), 2.34 (dd, 1H, H-2), 2.11 (s, 3H, H-27), 2.00 (m, 2H, H-14), 1.78 (m, 1H), 1.53 (m, 1H), 1.38 (m, 3H), 1.25 (s, 3H, H-22 or H-23+m, 1H), 1.10 (m, 1H), 0.96 (d, 3H, H-24), 0.90 (s, 3H, H-22 or H-23), 0.84 (d, 3H, H-25).

ESI-MS: 631.2, 80%; 633.2, 30% [M+H]⁺; 653.1, 100%; 655.1, 42% [M+Na]⁺. HRMS (monoisotopic): m/z 653.2423 [M+Na]⁺ calcd. for [C₃₃H₄₃ClN₂O₆S+Na] 653.2428.

5j: A. Preparation of the iminoester hydrochloride: A solution of 5g (42.7 mmole) m-tolunitrile in 40 ml EtOH/50 ml DEE is treated with HCl gas at Q 0° C. until saturation. The solution is then kept at RT over night, the solvent removed by evaporation and the residue dried in vacuo to provide 8.60 g of the crude iminoester hydrochloride. (¹H-NMR (400 MHz, DMSO-d₆): δ=7.92 (m, 2H), 7.60 (d, 1H), 7.50 (t, 1H), 4.60 (q, 2H), 2.37 (s, 3H), 1.47 (t, 3H)).

B. Oxazoline formation: To a solution of amino alcohol 3 (hydroacetate; 25.5 mg, 0.05 mmole) in 300 μL of DCE are added 30.0 mg (0.15 mmole) of the above iminoester hydrochloride and 20 μL of EtOH and the mixture is heated to 90° C. for 3 h. After cooling to RT the reaction mixture is directly submitted to FC in 3% MeOH/DCM to yield 23.2 mg of slightly impure product that is re-purified by FC in 2% MeOH/DCM to give 20.8 mg (68%) of the title compound 5j.

¹H-NMR (500 MHz, DMSO-d₆): δ=7.68 (s, 1H), 7.66 (t, 1H), 7.34 (m, 3H), 6.57 (s, 1H, H-17), 5.43 (dd, 1H, H-15), 5.16 (d, 1H, 3-OH), 4.55 (t (br), 1H, H-13), 4.31 (d, 1H, 7-OH), 4.20 (m, 1H, H-3), 3.82 (m, 1H, H-12), 3.51 (m, 1H, H-7), 3.21 (m, 1H, H-6), 2.64 (s, 3H, H-21), 2.45 (dd, 1H, H-2), 2.35 (s, 3H), 2.32 (m, 1H, H-2), 2.11 (s, 3H, H-27), 1.99 (m, 2H, H-14), 1.70 (m, 1H), 1.55 (m, 1H), 1.40 (m, 3H), 1.25 (s, 3H, H-22 or H-23+m, 1H), 1.15 (m, 1H), 0.95 (d, 3H, H-24), 0.89 (s, 3H, H-22 or H-23), 0.86 (d, 3H, H-25).

ESI-MS: 610.9 [M+H]⁺. HRMS: m/z 633.2978 [M+Na]⁺ calcd. for [C₃₄H₄₆N₂O₆S+Na] 633.2974.

5k: A. Preparation of the iminoester hydrochloride: A solution of 1.0 g (7.3 mmole) of 2-chloro benzonitrile in 8 ml EtOH/10 ml DEE is treated with HCl gas at 0° C. until saturation. The solution is then kept at RT over night, the solvent removed by evaporation and the residue dried in vacuo to provide 0.089 g of impure imino ester hydrochloride. No attempts are made to purify this material, which is directly used in the next step. (¹H-NMR (400 MHz, DMSO-d₆): δ=7.78 (d, 1H), 7.71 (d, 2H), 7.56 (m, 1H), 4.63 (q, 2H), 1.44 (t, 3H); additional peaks in the aromatic region of the spectrum).

B. Oxazoline formation: 33.0 mg (0.15 mmole) of the above iminoester hydrochloride are added to a solution of amino alcohol 3 (hydroacetate; 25.5 mg, 0.050 mmole) in 300 μL of DCE together with 20 μL of EtOH and the mixture is heated to 90° C. for 21 h. After cooling to RT the reaction mixture is directly submitted to FC in 2% MeOH/DCM to yield 20.97 mg of impure material that is further purified by preparative HPLC (30% 100% ACN in 100 min) to provide 6.08 mg (19%) of pure title compound 5k.

¹H-NMR (500 MHz, DMSO-d₆): δ=7.68 (d, 1H), 7.55 (m, 1H), 7.43 (t, 1H), 7.36 (s, 1H, H-19), 6.53 (s, 1H, H-17), 5.39 (d, 1H, H-15), 5.17 (d, 1H, 3-OH), 4.62 (t, 1H, H-13), 4.32 (d, 1H, 7-OH), 4.15 (m, 1H, H-3), 3.88 (m, 1H, H-12), 3.52 (m, 1H, H-7), 3.21 (m, 1H, H-6), 2.65 (s, 3H, H-21), 2.45 (dd, 1H, H-2), 2.34 (dd, 1H, H-2), 2.11 (s, 3H, H-27), 2.01 (m, 2H, H-14), 1.72 (t (br), 1H), 1.50 (m, 2H), 1.40 (m, 2H), 1.26 (s, 3H, H-22 or H-23+m, 1H), 1.13 (m, 1H), 0.96 (d, 3H, H-24), 0.89 (s, 3H, H-22 or H-23), 0.87 (d, 3H, H-25).

ESI-MS: 631, 100%; 633, 50% [M+H]⁺; 653, 75%; 655, 33% [M+Na]⁺. HRMS (monoisotopic): m/z 653.2430 [M+Na]⁺ calcd. for [C₃₃H₄₃ClN₂O₆S+Na] 653.2428.

5l: A. Preparation of the iminoester hydrochloride: A solution of 1.2 g (11.0 mmole) of thiophene-3-carbonitrile in 8 ml EtOH/10 ml DEE is treated with HCl gas at 0° C. until saturation. The solution is then kept at RT over night, evaporated to dryness and the residue dried in vacuo to provide 2.01 g of crude iminoester hydrochloride. (¹H-NMR (400 MHz, DMSO-d₆): δ=8.92 (m, 1H), 7.88 (d, 1H), 7.82 (dd, 1H), 4.57 (q, 2H), 1.46 (t, 3H)).

B. Oxazoline formation: 29.0 mg (0.15 mmole) of the above iminoester hydrochloride are added to a solution of amino alcohol 3 (hydroacetate; 25.5 mg, 0.05 mmole) in 300 μL of DCE together with 20 μL of EtOH and the mixture is heated to 90° C. for 4 h. After cooling to RT the reaction mixture is directly subjected to FC in 2% MeOH/DCM to yield 17.7 mg (59%) of pure title compound 51.

¹H-NMR (500 MHz, DMSO-d₆): δ=8.04 (s, 1H), 7.62 (m, 1H), 7.42 (d, 1H), 7.35 (s, 1H, H-19), 6.55 (s, 1H, H-17), 5.41 (dd, 1H, H-15), 5.15 (d, 1H, 3-OH), 4.51 (t, 1H, H-13), 4.30 (d, 1H, 7-OH), 4.21 (m, 1H, H-3), 3.78 (m, 1H, H-12), 3.51 (m, 1H, H-7), 3.21 (m, 1H, H-6), 2.65 (s, 3H, H-21), 2.46 (dd, 1H, H-2), 2.32 (dd, 1H, H-2), 2.10 (s, 3H, H-27), 1.97 (m, 2H, H-14), 1.70 (m, 1H), 1.52 (m, 1H), 1.40 (m, 3H), 1.25 (s, 3H, H-22 or H-23+m, 1H), 1.12 (m, 1H), 0.95 (d, 3H, H-24), 0.89 (s, 3H, H-22 or H-23), 0.85 (d, 3H, H-25).

ESI-MS: 602.8 [M+H]⁺. HRMS: m/z 625.2381 [M+Na]⁺ calcd. for [C₃₃H₄₂N₂O₆S₂+Na] 625.2382.

6a: A solution of 5g of 4-cyano-pyridine (48 mmole) in 40 ml EtOH/50 ml DEE is saturated with HCl gas at 0° C. (formation of a suspension). After storage of the mixture at RT over night the precipitate is collected by filtration (9.34 g). 2 g of this material (10 mmole, based on the molecular weight of the iminoester monohydrochloride) is redissolved in 20 ml of EtOH and treated with Et₃N (1 ml) for 18 h. The mixture is then diluted with DEE (60 ml) and insoluble material is removed by filtration and ished with DEE. The combined filtrates are evaporated to dryness to provide 1.4 g of a solid which, according to NMR, contained ca. 60% of the desired orthoester. This material is used in the next step without further purification.

Oxazoline formation: To a solution of amino alcohol 3 (hydroacetate; 25.5 mg, 0.05 mmole) in 300 μL of DCE are added 28.0 mg of the impure orthoester (0.15 mmole, based on the molecular weight of the orthoester) and 20 μL of EtOH and the mixture is heated to 90° C. for 3 h. After cooling to RT the reaction mixture is directly submitted to FC in 4% MeOH/DCM to yield 10.5 mg (35%) of the title compound 6a.

¹H-NMR (500 MHz, DMSO-d₆): δ=8.72 (d, 2H), 7.77 (d, 2H), 7.36 (s, 1H, H-19), 6.57 (s, 1H, H-17), 5.54 (m, 1H, H-15), 5.16 (d, 1H, 3-OH), 4.64 (m, 1H, H-13), 4.29 (d, 1H, 7-OH), 4.19 (m, 1H, H-3), 3.90 (m, 1H, H-12), 3.52 (m, 1H, H-7), 3.20 (m, 1H, H-6), 2.64 (s, 3H, H-21), 2.46 (dd, 1H, H-2), 2.34 (dd, 1H, H-2), 2.11 (s, 3H, H-27), 2.02 (m, 2H, H-14), 1.81 (m, 1H), 1.53 (m, 1H), 1.40 (m, 3H), 1.26 (s, 3H, H-22 or H-23+m, 1H), 1.13 (m, 1H), 0.95 (d, 3H, H-24), 0.90 (s, 3H, H-22 or H-23), 0.84 (d, 3H, H-25).

ESI-MS: 597.8 [M+H]⁺. HRMS: m/z 620.2765 [M+Na]⁺ calcd. for [C₃₄H₄₆N₂O₆S+Na] 620.2770.

6b: A solution of 5 g of 3-cyano-pyridine (48 mmole) in 40 ml EtOH/50 ml DEE is saturated with HCl gas at 0° C. (formation of a suspension). After storage of the mixture at RT over night the precipitate is collected by filtration (10.37 g). 2 g of this material (10 mmole, based on the molecular weight of the iminoester monohydrochloride) is redissolved in 20 ml of EtOH and treated with Et₃N (0.7 ml, 5 mmole) for 64 h. The mixture is then diluted with DEE (60 ml) and insoluble material is removed by filtration and ished with DEE. The combined filtrates are evaporated to dryness to provide 1.18 g of a solid, which is used in the next step without further purification.

Oxazoline formation: To a solution of amino alcohol 3 (hydroacetate; 25.5 mg, 0.050 mmole) in 300 μL of DCE are added 45.0 mg of material obtained in the above reaction sequence (0.150 mmole, based on the molecular weight of the orthoester) and 20 μL of EtOH and the mixture is heated to 90° C. for 3 h. After cooling to RT the reaction mixture is directly submitted to FC in 4% MeOH/DCM to yield 13.4 mg of impure material, which is further purified by preparative HPLC (30%→100% ACN in 100 min) to yield 9.93 mg (33%) of pure title compound 6b.

¹H-NMR (500 MHz, DMSO-d₆): δ=9.01 (d, 1H), 8.71 (d, 1H), 8.19 (dt, 1H), 7.52 (dd, 1H), 7.36 (s, 1H, H-19), 6.57 (s, 1H, H-17), 5.44 (t (br), 1H, H-15), 5.15 (d, 1H, 3-OH), 4.61 (m, 1H, H-13), 4.29 (d, 1H, 7-OH), 4.20 (m, 1H, H-3), 3.87 (m, 1H, H-12), 3.52 (m, 1H, H-7), 3.21 (m, 1H, H-6), 2.64 (s, 3H, H-21), 2.46 (dd, 1H, H-2), 2.33 (dd, 1H, H-2), 2.11 (s, 3H, H-27), 2.01 (m, 2H, H-14), 1.82 (m, 1H), 1.55 (m, 1H), 1.42 (m, 3H), 1.25 (s, 3H, H-22 or H-23+m, 1H), 1.17 (m, 1H), 0.95 (d, 3H, H-24), 0.89 (s, 3H, H-22 or H-23), 0.86 (d, 3H, H-25).

ESI-MS: 598.2 [M+H]⁺. HRMS: m/z 620.2766 [M+Na]⁺ calcd. for [C₃₄H₄₆N₂O₆S+Na] 620.2770.

6c: A mixture of 5.5 g (53 mmole) of 2-cyano pyridine and 2.67 g (58 mmole) of EtOH is treated with HCl gas, which resulted in almost immediate solidification of the reaction mixture. After addition of 12 ml of EtOH treatment with HCl gas is continued until saturation and the suspension is kept at 5° C. for 18 h. The precipitate is then isolated by filtration; this material proved to be a mixture of compounds of which the desired iminoester is only a minor component (based on signals for the ethyl ester group). A suspension of 3 g this mixture in 10 ml of EtOH is treated with 0.2 ml of Et₃N at RT for 18 h. The mixture is then filtered and the filtrate evaporated to dryness.

Qxazoline formation: 33.8 mg of the residue thus obtained are added to a solution of amino alcohol 3 (hydroacetate; 25.5 mg, 0.050 mmole) in 300 μL of DCE together with 10 μL of EtOH and the mixture is heated to 90° C. for 3 h. At this point additional material obtained in the above steps (50 mg) is added together with 50 μL of EtOH and heating at 90° C. is continued for further 21 h. After cooling to RT the reaction mixture is directly submitted to FC in 3% MeOH/DCM→5% MeOH/DCM to yield 2.26 mg of a ca. 7/1 mixture of 6c and the corresponding hydrolysis product (molecular mass of +18). Purification of this material by preparative HPLC (30%→100% ACN in 100 min) gives 1.02 mg of the title compound 6c. ESI-MS: 598.2 [M+H]⁺. Single peak on analytical HPLC (30%→100% ACN in 100 min; Rₜ=6.94 min).

6d: A. Preparation of the iminoester hydrochloride: A solution of 1.0 g (11.0 mmole) of 2-chloro-4-cyano pyridine in 8 ml EtOH/10 ml DEE is treated with HCl gas at 0° C. until saturation and the resulting suspension is stirred at RT over night. The mixture is then evaporated to dryness and the residue dried in vacuo to provide 1.39 g of the crude iminoester hydrochloride, which is used in the subsequent step without further purification. (¹H-NMR (400 MHz, DMSO-d₆): δ=8.69 (d, 1H), 8.13 (s, 1H), 7.97 (d, 1H), 4.55 (q, 2H), 1.45 (t, 3H).

B. Oxazoline formation: 33.0 mg (0.15 mmole) of the above iminoester hydrochloride are added to a solution of amino alcohol 3 (hydroacetate; 25.5 mg, 0.05 mmole) in 300 μL of DCE together with 20 μL of EtOH and the mixture is heated to 90° C. for 8 h. After cooling to RT the reaction mixture is directly submitted to FC in 3% MeOH/DCM to yield 15.91 mg (50%) of the title compound 6d.

¹H-NMR (500 MHz, DMSO-d₆): δ=8.57 (d, 1H), 7.82 (s (br), 1H), 7.81 (s (br), 1H), 7.38 (s, 1H, H-19), 6.60 (s, 1H, H-17), 5.48 (m, 1H, H-15), 5.18 (d (br), 1H, 3-OH), 4.69 (m, 1H, H-13), 4.31 (d (br), 1H, 7-OH), 4.19 (m, 1H, H-3), 3.95 (m, 1H, H-12), 3.52 (m, 1H, H-7), 3.22 (m, 1H, H-6), 2.67 (s, 3H, H-21), 2.47 (dd, 1H, H-2), 2.35 (dd, 1H, H-2), 2.11 (s, 3H, H-27), 2.05 (m, 2H, H-14), 1.71 (m, 1H), 1.57-1.10 (m, 6H), 1.28 (s, 3H, H-22 or H-23), 0.98 (d, 3H, H-24), 0.92 (s, 3H, H-22 or H-23), 0.86 (d, 3H, H-25).

ESI-MS: 631.8, 100% [M+H]⁺; 633.8, 39% [M+H]⁺. HRMS (monoisotopic): m/z 654.2381 [M+Na]⁺ calcd. for [C₃₂H₄₂ClN₃O₆S+Na] 654.2381.

6e: A solution of 1.25 g (9.03 mmole) of 6-chloro-3-cyano-pyridine in 10 ml EtOH/12 ml DEE is treated with HCl gas at 0° C. until saturation and the resulting suspension is stored at RT over night. The precipitate is isolated by filtration and triturated with a mixture of 10 ml EtOH/30 ml DEE to provide 1.23 g of crude iminoester hydrochloride, which is not further purified.

Oxazoline formation: 33.0 mg of this material ((0.15 mmole, based on the molecular weight of the iminoester mono hydrochloride) are then added to a solution of amino alcohol 3 (hydroacetate; 25.5 mg, 0.050 mmole) in 300 μL of DCE together 20 μL of EtOH and the mixture is heated to 90° C. for 8 h. After cooling to RT, the reaction mixture is directly submitted to FC in 4% MeOH/DCM to yield 14.27 mg of a slightly impure product, which is further purified by preparative HPLC (30%→100% ACN in 100 min) to give 11.8 mg (37%) of the title compound 6e.

¹H-NMR (500 MHz, DMSO-d₆): δ=8.83 (d, 1H), 8.25 (dd, 1H), 7.65 (dd, 1H), 7.35 (s, 1H, H-19), 6.57 (s, 1H, H-17), 5.44 (t (br), 1H, H-15), 5.16 (d (br), 1H, 3-OH), 4.64 (m, 1H, H-13), 4.30 (d (br), 1H, 7-OH), 4.18 (m, 1H, H-3), 3.89 (m, 1H, H-12), 3.50 (m, 1H, H-7), 3.20 (m, 1H, H-6), 2.64 (s, 3H, H-21), 2.45 (dd, 1H, H-2), 2.32 (dd, 1H, H-2), 2.11 (s, 3H, H-27), 2.02 (m, 2H, H-14), 1.80 (m, 1H), 1.55 (m, 1H), 1.43 (m, 3H), 1.25 (s, 3H, H-22 or H-23+m, 1H), 1.16 (m, 1H), 0.95 (d, 3H, H-24), 0.89 (s, 3H, H-22 or H-23), 0.86 (d, 3H, H-25).

ESI-MS: 631.8, 100% [M+H]⁺; 633.8, 39% [M+H]⁺. HRMS (monoisotopic): m/z 654.2380 [M+Na]⁺ calcd. for [C₃₂H₄₂ClN₃O₆S+Na] 654.2381.

6f: A solution of 500 mg ( ) of 6-bromo-3-cyano-pyridine (prepared from 6-chlor-3-cyano-pyridine by treatment with PBr₃ according to ref.) in 4 ml EtOH/5 ml DEE is treated with HBr gas at 0° C. for 30 min. (The use of HCl led to Br/Cl exchange). The mixture is then allowed to warm to RT and the solvent is evaporated to give 895 mg of crude iminoester hydrobromide. This material is suspended in 10 ml of EtOH and treated with 0.82 ml ( ) of Et₃N for 18 h. 15 ml of DEE are then added to the solution and the resulting precipitate is collected by filtration and dried (418 mg).

Oxazoline formation: 40.0 mg of the above material (0.15 mmole, based on the molecular weight of the iminoester free base) are added to a solution of amino alcohol 3 (hydroacetate; 25.5 mg, 0.050 mmole) in 300 μL of DCE together with 30 μL of EtOH and the mixture is heated to 90° C. After 3 h additional iminoester is added (18 mg) and heating is continued for two more hours. After cooling to RT, the reaction mixture is directly submitted to FC in 3% MeOH/DCM to yield 20.7 mg of slightly impure material ((>95%), which is further purified by preparative HPLC (5% 100% ACN in 100 min) to give, after lyophilization, 16.22 mg (48%) of pure title compound 6f as a white powder.

¹H-NMR (500 MHz, DMSO-d₆): δ=8.79 (d, 1H), 8.11 (dd, 1H), 7.78 (d, 1H), 7.35 (s, 1H, H-19), 6.57 (s, 1H, H-17), 5.44 (m, 1H, H-15), 5.16 (d, 1H, 3-OH), 4.64 (m, 1H, H-13), 4.29 (d (br), 1H, 7-OH), 4.18 (m, 1H, H-3), 3.88 (m, 1H, H-12), 3.50 (m, 1H, H-7), 3.20 (m, 1H, H-6), 2.64 (s, 3H, H-21), 2.45 (dd, 1H, H-2), 2.33 (dd, 1H, H-2), 2.11 (s, 3H, H-27), 2.02 (m, 2H, H-14), 1.81 (m, 1H), 1.55 (m, 1H), 1.38 (m, 3H), 1.25 (s, 3H, H-22 or H-23+m, 1H), 1.15 (m, 1H), 0.95 (d, 3H, H-24), 0.89 (s, 3H, H-22 or H-23), 0.84 (d, 3H, H-25).

ESI-MS: 676.1, 95% [M+H]⁺; 678.1, 100% [M+H]⁺. HRMS: m/z 676.20541 [M+H]⁺ calcd. for [C₃₂H₄₂BrN₃O₆S+H] 676.20561; 698.18691 [M+Na]⁺ calcd. for [C₃₂H₄₂BrN₃O₆S+Na] 698.18755.

6g: A solution of 1 g of 6-methyl-3-cyano-pyridine in 8 ml EtOH/10 ml DEE is treated with HCl gas at 0° C. until saturation and the resulting suspension is stored at RT over night. The mixture is evaporated to dryness and briefly dried to give 1.89 g of crude iminoester hydrochloride. 1.86 g of this material is suspended in 20 ml of EtOH, Et₃N (2 ml) is added, and the mixture is stirred at RT for 16 h (formation of a clear solution within 10 min after Et₃N addition). DEE (30 ml) is then added and the resulting precipitate is removed by filtration and ished with DEE. The combined filtrates are evaporated to dryness to provide 1.52 g of material, which according to NMR contained both the iminoester free base as well as the corresponding orthoester.

Oxazoline formation: 30.0 mg of the above material (0.15 mmole, based on the molecular weight of the iminoester free base) are then added to a solution of amino alcohol 3 (hydroacetate; 25.5 mg, 0.05 mmole) in 300 μL of DCE and the mixture is heated to 90° C. for 3 h. After cooling to RT the reaction mixture is directly submitted to FC in 4% MeOH/DCM to yield 22.3 mg of slightly impure (>90% purity) material, which is further purified by preparative HPLC (5%→100% ACN in 100 min) to give, after lyophilization, 16.2 mg (53%) of pure title compound 6g as a white powder.

¹H-NMR (500 MHz, DMSO-d₆): δ=8.87 (s, 1H), 8.06 (dd, 1H), 7.78 (d, 1H), 7.37 (s, 1H, H-19), 6.57 (s, 1H, H-17), 5.43 (m, 1H, H-15), 5.17 (d, 1H, 3-OH), 4.59 (m, 1H, H-13), 4.33 (d, 1H, 7-OH), 4.20 (m, 1H, H-3), 3.84 (m, 1H, H-12), 3.50 (m, 1H, H-7), 3.20 (m, 1H, H-6), 2.64 (s, 3H, H-21), 2.45 (dd, 1H, H-2), 2.32 (dd, 1H, H-2), 2.11 (s, 3H, H-27), 2.00 (m, 2H, H-14), 1.81 (t (br), 1H), 1.52 (m, 1H), 1.40 (m, 3H), 1.25 (s, 3H, H-22 or H-23+m, 1H), 1.13 (m, 1H), 0.94 (d, 3H, H-24), 0.89 (s, 3H, H-22 or H-23), 0.84 (d, 3H, H-25).

ESI-MS: 611.9 [M+H]⁺. HRMS: m/z 612.3102 [M+H]⁺ calcd. for [C₃₃H₄₅N₃O₆S+H] 612.3107.

6h: A. Preparation of 6-ethyl-3-cyano-pyridine: To a solution of 6-chloro-3-cyano-pyridine (277 mg, 2.0 mmole) and 35.3 mg Fe(acac)₂ (0.1 mmole) in 12 ml of THF and 1.13 ml of NMP is added 0.8 ml of a solution of EtMgBr in DEE (3 M; 2.4 mmole). The mixture is stirred at RT for 30 min at which point additional DEE (30 ml) is added followed by water (10 ml). The organic layer is separated and the aqueous solution is exhaustively extracted with DEE. The combined organic extracts are ished with water (2×10 ml), dried over MgSO₄ and the solvent is evaporated. The residue is purified by FC in hexane/AcOEt 4/1 to give 142 mg (54%) of 6-ethyl-3-cyano-pyridine. (ESI-MS: 133 [M+H]⁺. ¹H-NMR (400 MHz, CDCl₃): δ=8.80 (s, 1H), 7.85 (dd, 1H), 7.29 (d, 1H), 2.90 (q, 2H), 1.25 (t, 3H)).

B. A solution of 140 mg of 6-ethyl-3-cyano-pyridine (1.06 mmole) in 4 ml EtOH/5 ml DEE is treated with HCl gas until saturation (intermittent formation of a suspension, but after saturation a clear solution had formed). The mixture is kept at RT over night, the solvent is evaporated and the residue is briefly dried in vacuo (228 mg). This material is then redissolved in 5 ml of EtOH and Et₃N (0.4 mL) is added. After 30 min DEE (25 ml) is added, the resulting precipitate is removed by filtration and ished with DEE, and the combined filtrates are evaporated to dryness to yield 133 mg of crude material that is used in the next step without further purification. 26.7 mg of the material (0.15 mmole, based on the molecular weight of the iminoester free base) are added to a solution of amino alcohol 3 (hydroacetate; 25.5 mg, 0.05 mmole) in 300 μL of DCE together with 30 μL of EtOH and the mixture is heated to 90° C. for 6 h. Additional iminoester/orthoester is added after 6 h (15 mg) and 9 h (20 mg). After a total of 19 h at 90° C. the reaction mixture is directly submitted to FC in 5% MeOH/DCM to yield 6.6 mg (21%) of an essentially pure product. For biological studies the material is further purified by preparative HPLC (5% 100% ACN in 100 min) to give, after lyophilization, 2.8 mg of pure title compound 6h as a white powder.

¹H-NMR (500 MHz, DMSO-d₆): δ=8.88 (d, 1H), 8.06 (dd, 1H), 7.37 (d, 1H), 7.33 (d, 1H, H-19), 6.55 (s, 1H, H-17), 5.42 (m, 1H, H-15), 5.15 (d, 1H, 3-OH), 4.58 (m, 1H, H-13), 4.29 (d, 1H, 7-OH), 4.20 (m, 1H, H-3), 3.84 (m, 1H, H-12), 3.50 (m, 1H, H-7), 3.20 (m, 1H, H-6), 2.79 (q, 2H, Et oxazoline), 2.64 (s, 3H, H-21), 2.47 (dd, 1H, H-2), 2.32 (dd, 1H, H-2), 2.11 (s, 3H, H-27), 2.00 (m, 2H, H-14), 1.70 (t (br), 1H), 1.53 (m, 1H), 1.40 (m, 3H), 1.25 (s, 3H, H-22 or H-23+m, 1H), 1.23 (t, 3H, Et oxazoline), 1.15 (m, 1H), 0.96 (d, 3H, H-24), 0.90 (s, 3H, H-22 or H-23), 0.86 (d, 3H, H-25).

ESI-MS: 625.9 [M+H]⁺. HRMS: m/z 626.3259 [M+H]⁺ calcd. for [C₃₄H₄₇N₃O₆S+H] 626.3264.

6i: A. Preparation of 6-iso-propyl-3-cyano-pyridine: To a solution of 6-chloro-3-cyano-pyridine (277 mg, 2.0 mmole) and 35.3 mg Fe(acac)₂ (0.1 mmole) in 12 ml of THF and 1.13 ml of NMP are added 2.4 ml of a solution of iso-C₃H₇MgBr in DEE (1 M; 2.4 mmole). The mixture is stirred at RT for 30 min at which point additional DEE (30 ml) is added followed by water (10 ml). The organic layer is separated and the aqueous solution is exhaustively extracted with DEE. The combined organic extracts are ished with water (2×10 ml), dried over MgSO₄ and the solvent is evaporated. The residue is purified by FC in hexane/AcOEt 4/1 to give 224 mg (76%) of 6-iso-propyl-3-cyano-pyridine. (ESI-MS: 146.9 [M+H]⁺. ¹H-NMR (400 MHz, CDCl₃): δ=8.80 (s, 1H), 7.85 (dd, 1H), 7.29 (d, 1H), 3.13 (quin, 1H), 1.33 (d, 6H)).

B. 200 mg (1.36 mmole) of 6-iso-propyl-3-cyano-pyridine are converted into 274 mg of a crude mixture of the corresponding iminoester free base and orthoester as described for 6h. 30.0 mg of this material (0.15 mmole, based on the molecular weight of the iminoester free base) are added to a solution of amino alcohol 3 (hydroacetate; 25.5 mg, 0.05 mmole) in 300 μL of DCE together with 30 μL of EtOH and the mixture is heated to 90° C. for 19 h. After cooling to RT the reaction mixture is directly submitted to FC in 5% MeOH/DCM to yield 6.2 mg (19%) of an essentially pure product. For biological studies the material is further purified by preparative HPLC (5%→100% ACN in 100 min) to give, after lyophilization, 4.3 mg of pure title compound 6i as a white powder.

¹H-NMR (500 MHz, DMSO-d₆): δ=8.92 (s, 1H), 8.09 (d, 1H), 7.38 (d, 1H), 7.35 (d, 1H, H-19), 6.55 (s, 1H, H-17), 5.42 (m, 1H, H-15), 5.17 (d, 1H, 3-OH), 4.61 (m, 1H, H-13), 4.34 (d, 1H, 7-OH), 4.20 (m, 1H, H-3), 3.83 (m, 1H, H-12), 3.51 (m, 1H, H-7), 3.20 (m, 1H, H-6), 3.13 (quin, 1H, i-propyl oxazoline), 2.66 (s, 3H, H-21), 2.48 (dd, 1H, H-2), 2.34 (dd, 1H, H-2), 2.10 (s, 3H, H-27), 1.99 (m, 2H, H-14), 1.79 (m, 1H), 1.52 (m, 1H), 1.38 (m, 3H), 1.25 (s, H-22 or H-23+2×d+m, 10H), 1.12 (m, 1H), 0.94 (d, 3H, H-24), 0.89 (s, 3H, H-22 or H-23), 0.86 (d, 3H, H-25).

ESI-MS: 639.9 [M+H]⁺. HRMS: m/z 640.3424 [M+H]⁺ calcd. for [C₃₅H₄₉N₃O₆S+H] 640.3420.

6j: 1.16 g of (7.25 mmole) of 6-tert.-butyl-3-cyano-pyridine (prepared from 3-cyano-pyridine and pivalic acid according to ref. 3) are converted into 1.26 g of a crude mixture of iminoester free base and the corresponding orthoester as described for 6h. 30.0 mg of this material (0.15 mmole, based on the molecular weight of the iminoester free base) are added to a solution of amino alcohol 3 (hydroacetate; 25.5 mg, 0.050 mmole) in 300 μL of DCE and the mixture is heated to 90° C. for 1 h. After cooling to RT the reaction mixture is directly submitted to FC in 5% MeOH/DCM to yield 33.2 mg of impure material, which is further purified by preparative HPLC (5%→100% ACN in 100 min) to give, after lyophilization, 18.5 mg (57%) of pure title compound 6j as a white powder.

¹H-NMR (500 MHz, DMSO-d₆): δ=8.92 (d, 1H), 8.09 (d, 1H), 7.51 (d, 1H), 7.33 (d, 1H, H-19), 6.55 (s, 1H, H-17), 5.42 (m, 1H, H-15), 5.14 (d, 1H, 3-OH), 4.59 (m, 1H, H-13), 4.30 (d, 1H, 7-OH), 4.20 (m, 1H, H-3), 3.85 (m, 1H, H-12), 3.51 (m, 1H, H-7), 3.21 (m, 1H, H-6), 2.64 (s, 3H, H-21), 2.46 (dd, 1H, H-2), 2.34 (dd, 1H, H-2), 2.11 (s, 3H, H-27), 1.99 (m, 2H, H-14), 1.77 (m, 1H), 1.50 (m, 1H), 1.37 (m, 3H), 1.33 (s, 9H, tert.-butyl oxazoline), 1.26 (s, 3H-22 or H-23+m, 1H), 1.11 (m, 1H), 0.96 (d, 3H, H-24), 0.90 (s, 3H, H-22 or H-23), 0.85 (d, 3H, H-25). ESI-MS: 654.0 [M+H]⁺.

TABLE 1

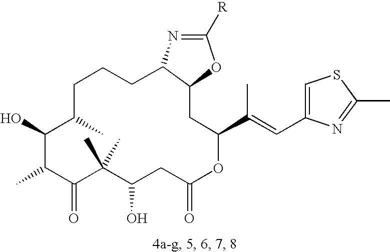

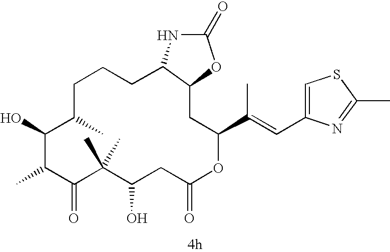

| | | Tub-Pol. | | |
|---|---|---|---|---|
| | | (5 μM/ | IC₅₀KB-31 | IC₅₀KB-8511 |
| Compound | R | 2 μM) | (nM) | (nM) |
| 4a | CH₃ | | >1000 | >1000 |
| 4b | C₂H₅ | | 1857 | 5543 |
| 4c | n-C₄H₉ | | 344/386 | 923/998 |
| 4d | tert.-butyl | | 12 | 55 |
| 4e | OCH₃ | | 1266 | 5525 |
| 4f | 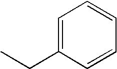 | | 940/1137 | 3763 |
| 4g | 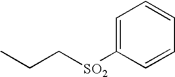 | | 3379 | 4037 |
| 4h | C═O | | 615/640 | >10,000 |
| 5a | 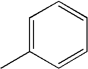 | 80/63 | 30 | 70 |
| 5b | 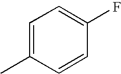 | | 27 | 50 |
| 5c | 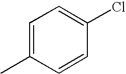 | 92/72 | 6 | 10 |
| 5d | 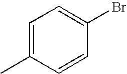 | | 3.9 | 7.2 |
| 5e | 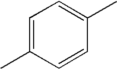 | | 8 | 13 |
| 5f | 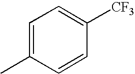 | | 65 | 80 |
| 5g | 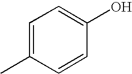 | | 3 | 133 |
| 5h | 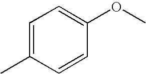 | | 110 | 140 |
| 5i | 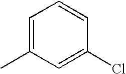 | 80/67 | 117 | 145 |
| 5j | 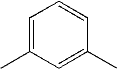 | | 118 | 180 |
| 5k | 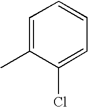 | | 113 | 180 |
| 5l | 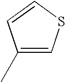 | | 42 | 146 |
| 6a | 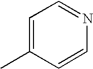 | | 30 | 83 |
| 6b | 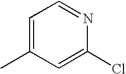 | | 330 | 497 |
| 6c | 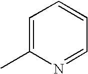 | | 24 | 370 |
| 6d | 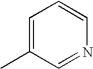 | 89/73 | 1.3 | 5.4 |
| 6e | 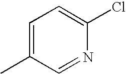 | 93/73 | 2.09/1.74 | 4.18/3.69 |
| 6f | 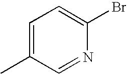 | | 1.65 | 3.34 |
| 6g | 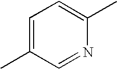 | | 2.64 | 6.75 |
| 6h | 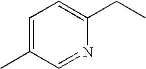 | | 66.7 | 162 |
| 6i | 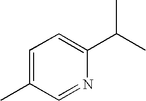 | | 480 | 582 |
| 6j | 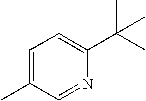 | | >1000 | >1000 |

### Chemical Biology

The biological activities of the synthesized epothilones are evaluated through tubulin polymerization assays.

In one assay, the fraction of tubulin polymerized into microtubules upon exposure to a given concentration of the respective compound is determined.

### Induction of Polymerisation

Induction of polymerization of pure bovine brain tubulin by 5 μM (2 μM) of test compound relative to the effect of 25 μM of Epothilone B, which gives maximal polymerization (100% value). Tubulin polymerization is determined using a centrifugation-based assay.

### Results

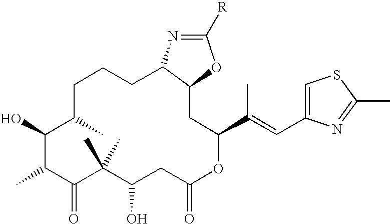

| Compound | Tub-Pol. |
|---|---|
| R = | (5 μM/2 μM) |
| 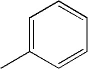 | 80/63 |
| 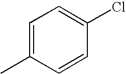 | 92/72 |
| 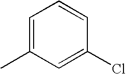 | 80/67 |
| 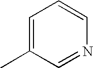 | 89/73 |
| 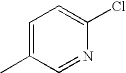 | 93/73 |

### Determination of IC₅₀ Values Against Human Epidermoid Carcinoma Cell Lines KB-31

IC₅₀-values for growth inhibition of the human epidermoid carcinoma cell lines KB-31 and KB-8511, respectively. KB-8511 is a P-glycoprotein 170 (P-gp170)-overexpressing multidrug-resistant subline of the KB-31 parental line. Cells are exposed to compounds for 72 h. Cell numbers are estimated by quantification of protein content of fixed cells by methylene blue staining.

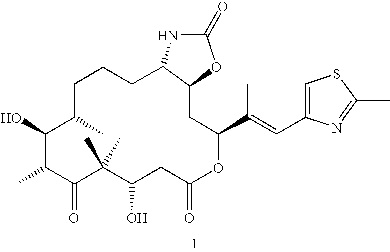

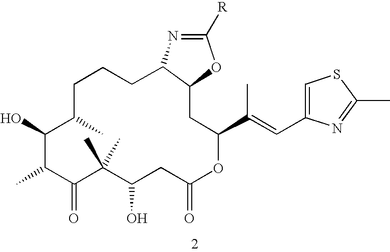

Results

| | IC50-KB-31 | IC50-KB-8511 |
|---|---|---|
| Compound | (nM) | (nM) |
| 1 | +++ | + |
| 2, R | | |
| H | + | + |
| CH₃ | + | + |
| C₂H₅ | + | + |
| n-C₄H₉ | ++++ | ++ |
| tert.-butyl | +++++ | +++++ |
| OCH₃ | + | + |
| 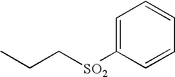 | + | + |
| 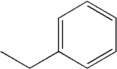 | + | + |
| 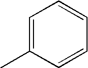 | ++++++ | ++++++ |
| 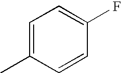 | ++++++ | ++++++ |
| 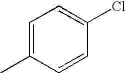 | ++++++ | ++++++ |
| 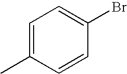 | ++++++ | ++++++ |
| 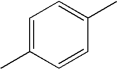 | ++++++ | ++++++ |
| 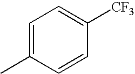 | ++++++ | ++++++ |
| 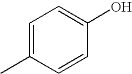 | ++++++ | +++++ |
| 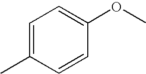 | +++++ | +++++ |
| 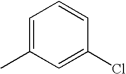 | +++++ | +++++ |
| 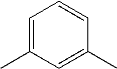 | +++++ | +++++ |
| 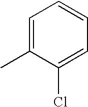 | +++++ | +++++ |
| 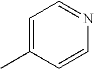 | ++++++ | ++++++ |
| 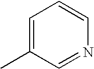 | ++++++ | ++++++ |
| 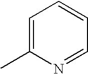 | ++++++ | ++++ |
| 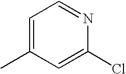 | ++++ | ++++ |
| 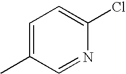 | ++++++ | ++++++ |
| 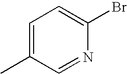 | ++++++ | ++++++ |
| 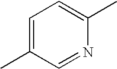 | ++++++ | ++++++ |
| 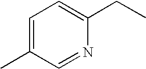 | ++++++ | +++++ |
| 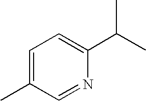 | ++++ | +++ |
| 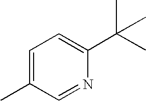 | + | + |
| 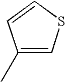 | ++++++ | +++++ |

Where:
+ >1000 nm
++ 1000-750 nm
+++ 750-500 nm
++++ 500-250 nm
+++++ 250-100 nm
++++++ <100 nm

1. A compound selected from the group consisting of formula A, B, I or II: 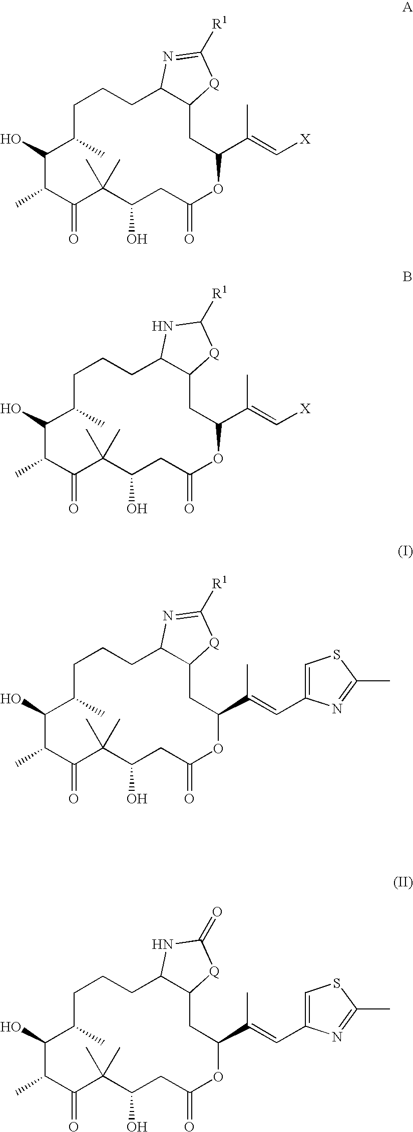
where Q is a radical selected from O or S; and
R may be selected from hydrogen, halogen, hydroxy, hydrocarbyl, trifluoromethyl, cyano, nitro, oxo, amidino, —B(OH)₂, ═NR², —OR², —SR², —C(O)R², —C(O)OR², —OC(O)R², —N(R²)R³, —C(O)_{K}N(R²)R³, (CR⁵R⁶)ⱼ—S(O)ₗR², —C(R²)₃ and R⁴;
R² and R³ are each independently hydrogen or are selected from C₁₋₆ alkyl, —(CR⁵R⁶)ⱼ-carbocyclyl and —(CR⁵R⁶)ⱼ-heterocyclyl, any of which is optionally substituted with 1, 2, 3, 4 or 5 substituents independently selected from halogen, hydroxy, C₁₋₃ alkyl, trifluoromethyl, cyano, nitro, amino and amidino;
R⁴ and X are each independently selected from C₁₋₆ alkyl, C₁₋₆ alkenyl, C₁₋₆ alkynyl, C₁₋₆ alkoxy, —(CR₅R⁶)ⱼ-carbocyclyl and —(CR⁵R⁶)ⱼ-heterocyclyl, aryl, heteroaryl, amino any of which is optionally substituted with 1, 2, 3, 4 or 5 substituents independently selected from halogen, hydroxy, C₁₋₆ alkyl and C₁₋₆ alkoxy;
each R⁵ and R⁶ may be the same or different and are both independently selected from a bond, hydrogen, halogen, hydroxy and amino;
j is 0, 1, 2, 3, 4, 5, 6 or 7;
K is 0 or 1;
l is 0, 1 or 2
or pharmaceutically acceptable salts, esters, N-oxides or prodrugs thereof.

2. A compound of claim 1, wherein Q is O.

3. A compound of claim 1, wherein R1 has the formula 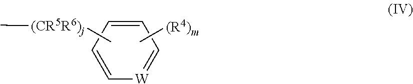
where R⁵, R⁶, j and R⁴ are as hereinbefore described; and
W is N or C; and
m is 0, 1, 2, 3 or 4.

4. A compound of claim 1, where R1 has the formula: 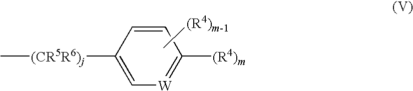
where R⁵, R⁶, j and R⁴ are as hereinbefore described; and
W is N or C; and
m is 0, 1, 2, 3 or 4.

5. A compound of claim 1, where R¹ has the formulae: 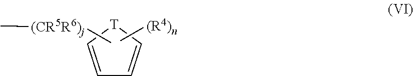
where n is 0, 1, 2 or 3 and T is NH, O or S.

6. A compound of claim 5, wherein n is 1.

7. A compound of claim 1, wherein m is 1.

8. A compound of claim 1, wherein R⁵ and R⁶ are both hydrogen.

9. A compound of claim 1, wherein j is 0.

10. A compound of claim 1, wherein R⁴ is selected from halogen and C₁₋₆ alkyl.

11. A compound of claim 1, where R¹ is selected from one of the following:
H, CH₃, C₂H₅, n-C₄H₉, tert.-butyl, OCH₃,

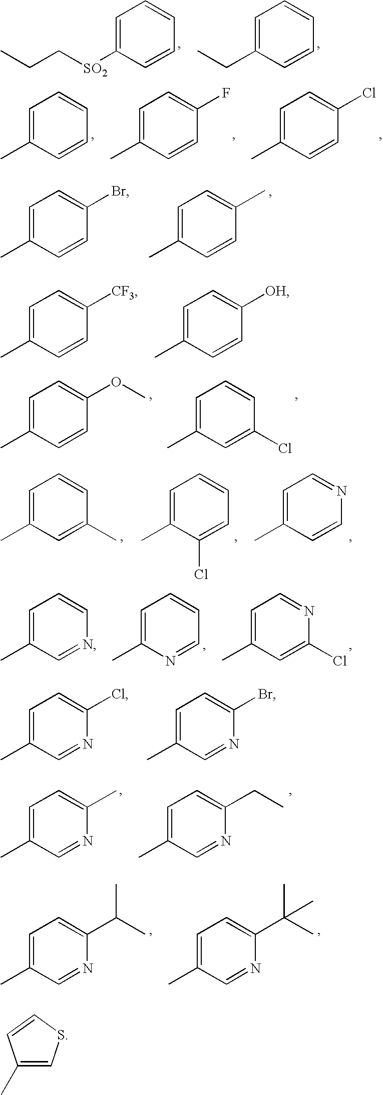

12. A pharmaceutical composition comprising a compound of claim 1 and at least one pharmaceutically acceptable carrier.

13-14. (canceled)

15. A method of treating cancer, which comprises administering a compound of claim 1, in an amount effective to treat that disease to a warm blooded animal.

16. A process for killing a cancer cell, which comprises contacting a cytotoxic solution comprising a compound of claim 1 to the cancer cell.

17. A process for the preparation of a compound of claim 1 comprising treating a compound of formula 3 with a) an orthoester or b) an iminoester

18. A compound of claim 1 wherein X is aryl or heteroaryl.
